SEQUENCE LISTING (4 sequences)

SEQ ID NO: 1 - RNA, 21 nt, Artificial Sequence
  siRNA sense
gucaucacacugaauaccaau

SEQ ID NO: 2 - RNA, 23 nt, Artificial Sequence
  siRNA antisense
auugguauucagugugaugacac

SEQ ID NO: 3 - DNA, 21 bp, Artificial Sequence
  siRNA sense
ggancancncaagncnnactt

SEQ ID NO: 4 - DNA, 21 bp, Artificial Sequence
  siRNA antisense
gnaagacnngagangancctt

(19) United States
(12) Patent Application Publication
(10) Pub. No.: US 2014/0294978 A1
(43) Pub. Date: Oct. 2, 2014

(54) CATIONIC LIPID

(71) Applicant: Kyowa Hakko Kirin Co., Ltd., Tokyo (JP)

(72) Inventors: Takeshi KUBOYAMA, Tokyo (JP); Kaori YAGI, Tokyo (JP); Tomoyuki NAOI, Tokyo (JP); Tomohiro ERA, Tokyo (JP)

(73) Assignee: Kyowa Hakko Kirin Co., Ltd., Tokyo (JP)

(21) Appl. No.: 14/206,193

(22) Filed: Mar. 12, 2014

Related U.S. Application Data
(60) Provisional application No. 61/789,466, filed on Mar. 15, 2013.

(51) Int. Cl. A61K 47/18; A61K 47/48

(52) U.S. Cl. CPC A61K 47/18; A61K 47/48046
USPC 424/490; 560/165; 548/567; 546/247; 514/44 R; 536/23.1; 514/44 A; 435/375

(57) ABSTRACT

The present invention provides a cationic lipid and the like. The cationic lipid is for delivering a medicament containing a cationic lipid which facilitates the introduction of a nucleic acid into a cell or the like, and is represented by formula (I).

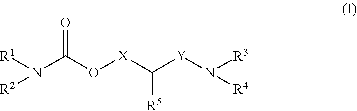

In the formula, R¹ is linear or branched alkyl, alkenyl, or alkynyl, each having 8 to 24 carbon atoms,
R² is linear or branched alkyl, alkenyl, or alkynyl, each having 8 to 24 carbon atoms, or alkoxyethylene, alkoxypropylene, alkenyloxyethylene, alkenyloxypropylene, alkynyloxyethylene, or alkynyloxypropylene,
R³ and R⁴ may be the same or different, and are each alkyl having 1 to 3 carbon atoms or are combined together to form alkylene having 2 to 6 carbon atoms, or R³ and R⁵ are combined together to form alkylene having 2 to 6 carbon atoms,
R⁵ is a hydrogen atom, alkyl having 1 to 6 carbon atoms, alkenyl having 3 to 6 carbon atoms, amino, monoalkylamino, hydroxy, alkoxy, carbamoyl, monoalkylcarbamoyl, dialkylcarbamoyl, or the like, or is combined together with R³ to form alkylene having 2 to 6 carbon atoms,
X is alkylene having 1 to 6 carbon atoms, and
Y is a single bond, alkylene having 1 to 6 carbon atoms, or the like.

### BACKGROUND OF THE INVENTION

1. Field of the Invention

The present invention relates to a novel cationic lipid which facilitates the introduction of a nucleic acid into, for example, a cell or the like; a novel composition containing the cationic lipid; and the like.

2. Background Art

A cationic lipid is an amphiphilic molecule having a lipophilic region containing one or more hydrocarbon groups and a hydrophilic region containing at least one positively charged polar head group. The formation of a complex which is positively charged as a whole between a cationic lipid and a macromolecule such as a nucleic acid facilitates the entry of the macromolecule such as a nucleic acid into a cytoplasm through a cell plasma membrane, and therefore, the cationic lipid is useful. This process, which can be performed in vitro and in vivo, is known as transfection.

Patent documents 1 and 2 disclose cationic lipids and lipid particles containing the lipid, which are advantageous for in vivo delivery of a nucleic acid and for a nucleic acid-lipid particle composition suitable for use in in vivo therapy. For example, patent document 1 discloses a cationic lipid such as

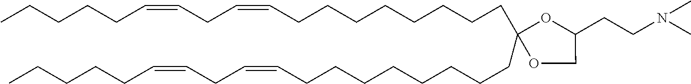 [SMILES: CCCCC/C=C\C/C=C\CCCCCCCCC1(CCCCCCCC/C=C\C/C=C\CCCCC)OCC(CCN(C)C)O1]

2,2-dilinoleyl-4-(2-dimethylaminoethyl)-[1,3]-dioxolane (DLin-KC2-DMA), and patent document 2 discloses a cationic lipid such as

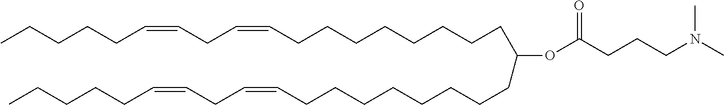 [SMILES: CCCCC/C=C\C/C=C\CCCCCCCCC(CCCCCCCC/C=C\C/C=C\CCCCC)OC(=O)CCCN(C)C]

(6Z,9Z,28Z,31Z)-heptatriaconta-6,9,28,31-tetraen-19-yl 4-(dimethylamino)butanoate (DLin-MC3-DMA).
Patent Document 1: WO02010/042877
Patent Document 2: WO2010/054401

### SUMMARY OF THE INVENTION

An object of the present invention is to provide a novel cationic lipid which facilitates the introduction of a nucleic acid into, for example, a cell or the like; a novel composition containing the cationic lipid; and the like.

The present invention relates to the following (1) to (20).

(1) A cationic lipid represented by formula (I):

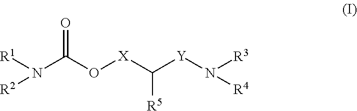

(Wherein R¹ is linear or branched alkyl, alkenyl, or alkynyl, each having 8 to 24 carbon atoms,
R² is linear or branched alkyl, alkenyl, or alkynyl, each having 8 to 24 carbon atoms, or alkoxyethylene, alkoxypropylene, alkenyloxyethylene, alkenyloxypropylene, alkynyloxyethylene, or alkynyloxypropylene,
R³ and R⁴ may be the same or different, and are each alkyl having 1 to 3 carbon atoms or are combined together to form alkylene having 2 to 6 carbon atoms, or R³ and R⁵ are combined together to form alkylene having 2 to 6 carbon atoms,
R⁵ is a hydrogen atom, alkyl having 1 to 6 carbon atoms, alkenyl having 3 to 6 carbon atoms, amino, monoalkylamino, hydroxy, alkoxy, carbamoyl, monoalkylcarbamoyl, dialkylcarbamoyl, or alkyl having 1 to 6 carbon atoms or alkenyl having 3 to 6 carbon atoms, each substituted with one to three of the same or different substituents selected from amino, monoalkylamino, hydroxy, alkoxy, carbamoyl, monoalkylcarbamoyl, and alkylcarbamoyl, or is combined together with R³ to form alkylene having 2 to 6 carbon atoms,
X is alkylene having 1 to 6 carbon atoms, and
Y is a single bond or alkylene having 1 to 6 carbon atoms, provided that the sum of the number of carbon atoms in X and Y is 6 or less, and when R⁵ is a hydrogen atom, Y is a single bond, and when R⁵ and R⁶ are combined together to form alkylene having 2 to 6 carbon atoms, Y is a single bond, or methylene or ethylene).
   (2) The cationic lipid according to the above (1), wherein R¹ and R² are tetradecyl, hexadecyl, (Z)-tetradec-9-enyl, (Z)-hexadec-9-enyl, (Z)-octadec-6-enyl, (Z)-octadec-9-enyl, (E)-octadec-9-enyl, (Z)-octadec-1-enyl, (9Z,12Z)-octadeca-9,12-dienyl, (9Z,12Z,15Z)-octadeca-9,12,15-trienyl, (Z)-icos-11-enyl, (11Z,14Z)-icosa-11,14-dienyl, 3,7,11-trimethyldodeca-2,6,10-trienyl, or (Z)-docos-13-enyl.
   (3) The cationic lipid according to the above (1), wherein R¹ and R² are hexadecyl, (Z)-hexadec-9-enyl, (Z)-octadec-6-enyl, (Z)-octadec-9-enyl, (9Z,12Z)-octadeca-9,12-dienyl, (Z)-icos-11-enyl, or (11Z,14Z)-icosa-11,14-dienyl.
   (4) The cationic lipid according to any one of the above (1) to (3), wherein X is alkylene having 1, 2, or 3 carbon atoms, and Y is a single bond or methylene.
   (5) The cationic lipid according to any one of the above (1) to (4), wherein R³ and R⁴ may be the same or different, and are each methyl or ethyl, or are combined together to form butylene or pentylene.
   (6) The cationic lipid according to any one of the above (1) to (4), wherein R³ and R⁵ are combined together to form propylene, butylene, or pentylene, and R⁴ is methyl or ethyl.
   (7) A composition containing the cationic lipid described in any one of the above (1) to (6) and a nucleic acid.
   (8) The composition according to the above (7), wherein the cationic lipid forms a complex between the cationic lipid and the nucleic acid, or forms a complex between a combination of a neutral lipid and/or a polymer with the cationic lipid and the nucleic acid.
   (9) The composition according to the above (7), wherein the cationic lipid forms a complex between the cationic lipid and the nucleic acid, or forms a complex between a combination of a neutral lipid and/or a polymer with the cationic lipid and the nucleic acid, and the composition contains the complex and a lipid membrane which encapsulates the complex.
   (10) The composition according to any one of the above (7) to (9), wherein the nucleic acid is a nucleic acid which has an activity of suppressing the expression of a target gene by utilizing RNA interference (RNAi).
   (11) The composition according to the above (10), wherein the target gene is a gene which is expressed in the liver, lung, kidney, or spleen.
   (12) A method for introducing the nucleic acid described in any one of the above (7) to (11) into a cell by using the composition described in any one of the above (7) to (11).
   (13) The method according to the above (12), wherein the cell is a cell which is present in the liver, lung, kidney, or spleen of a mammal.
   (14) The method according to the above (12) or (13), wherein the method for introduction into a cell is a method for introduction into a cell by intravenous administration.
   (15) A method for treating a disease associated with the liver, lung, kidney, or spleen, comprising administering the composition described in the above (11) to a mammal.
   (16) The method according to the above (1.5), wherein the method of administration is intravenous administration.
   (17) A pharmaceutical composition for use in the treatment of a disease, comprising the composition described in the above (10).
   (18) The pharmaceutical composition according to the above (17), which is for intravenous administration.
   (19) A therapeutic agent for a disease associated with the liver, lung, kidney, or spleen, which comprises the composition described in the above (11) and is for use in the treatment of a disease associated with the liver, lung, kidney, or spleen.
   (20) The therapeutic agent for a disease associated with the liver, lung, kidney, or spleen according to the above (19), which is for intravenous administration.

### Effect of the Invention

By administering a composition containing the novel cationic lipid of the present invention and a nucleic acid to a mammal or the like, the nucleic acid can be easily introduced into, for example, a cell or the like.

### BRIEF DESCRIPTION OF THE DRAWINGS

FIG. 1 shows the expression ratio of a target gene mRNA after the introduction of preparations obtained in Example 9 (preparations using any of Compounds I-1, and 1-3 to I-5) and preparations obtained in Comparative Example 1 (preparations using any of DLin-KC2-DMA and Compounds VII-1 to VII-3) into cells of a human liver cancer-derived cell line HepG2. The ordinate represents the expression ratio of the target gene mRNA when the expression level of a negative control was taken as 1; and the abscissa represents the nucleic acid concentration (nM), and the compound numbers of the cationic lipids used.

FIG. 2 shows the cholesterol level in blood at 48 hours after the administration of preparations obtained in Example 9 (preparations using any of Compounds I-1 to I-5) and a preparation obtained in Comparative Example 1 (a preparation using DLin-KC2-DMA) to mice, respectively, at a dose corresponding to 0.3 mg/kg of siRNA. The ordinate represents the relative value of the cholesterol level in blood when the cholesterol level in blood of a saline-administered group was taken as 100.

FIG. 3 shows the cholesterol level in blood at 48 hours after the administration of preparations obtained in Example 9 (preparations using any of Compounds I-1 to I-5) and preparations obtained in Comparative Example 1 (preparations using any of DLin-KC2-DMA and Compounds VII-1 to VII-3) to mice, respectively, at a dose corresponding to 3 mg/kg of siRNA. The ordinate represents the relative value of the cholesterol level in blood when the cholesterol level in blood of a saline-administered group was taken as 100.

FIG. 4 shows the Factor VII protein level in plasma at 48 hours after the administration of preparations obtained in Examples 10 and 11 (preparations using Compound I-6, I-1, I-7, or I-8) to mice, respectively, at a dose corresponding to 0.3 mg/kg of siRNA. The ordinate represents the relative value of the Factor VII protein level in plasma when the Factor VII protein level in plasma of a saline-administered group was taken as 100.

### DESCRIPTION OF THE EMBODIMENTS

The cationic lipid of the present invention is a cationic lipid represented by formula (I):

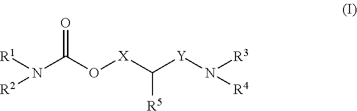

(wherein R¹ is linear or branched alkyl, alkenyl, or alkynyl, each having 8 to 24 carbon atoms,
R² is linear or branched alkyl, alkenyl, or alkynyl each having 8 to 24 carbon atoms, or alkoxyethylene, alkoxypropylene, alkenyloxyethylene, alkenyloxypropylene, alkynyloxyethylene, or alkynyloxypropylene,
R³ and R⁴ may be the same or different, and are each alkyl having to 3 carbon atoms or are combined together to form alkylene having 2 to 6 carbon atoms, or R³ and R⁵ are combined together to form alkylene having 2 to 6 carbon atoms,
R⁵ is a hydrogen atom, alkyl having 1 to 6 carbon atoms, alkenyl having 3 to 6 carbon atoms, amino, monoalkylamino, hydroxy, alkoxy, carbamoyl, monoalkylcarbamoyl, dialkylcarbamoyl, or alkyl having 1 to 6 carbon atoms or alkenyl having 3 to 6 carbon atoms, each substituted with one to three of the same or different substituents selected from amino, monoalkylamino, hydroxy, alkoxy, carbamoyl, monoalkylcarbamoyl, and alkylcarbamoyl, or is combined with R³ to form alkylene having 2 to 6 carbon atoms,
X is alkylene having 1 to 6 carbon atoms, and
Y is a single bond or alkylene having 1 to 6 carbon atoms, provided that the sum of the number of carbon atoms in X and Y is 6 or less, and when R⁵ is a hydrogen atom, Y is a single bond, and when R⁵ and R³ are combined together to form alkylene having 2 to 6 carbon atoms, Y is a single bond or methylene or ethylene).

Examples of the linear or branched alkyl having 8 to 24 carbon atoms include octyl, decyl, dodecyl, tridecyl, tetradecyl, 2,6,10-trimethylundecyl, pentadecyl, 3,7,11-trimethyldodecyl, hexadecyl, heptadecyl, octadecyl, 6,10,14-trimethylpentadecan-2-yl, nonadecyl, 2,6,10,14-tetramethylpentadecyl, icosyl, 3,7,11,15-tetramethylhexadecyl, henicosyl, docosyl, tricosyl, and the like.

The linear or branched alkenyl having 8 to 24 carbon atoms may be linear or branched alkenyl having 8 to 24 carbon atoms and having 1 to 3 double bonds. Examples thereof include (Z)-tetradec-9-enyl, (Z)-hexadec-9-enyl, (Z)-octadec-6-enyl, (Z)-octadec-9-enyl, (E)-octadec-9-enyl, (Z)-octadec-11-enyl, (9Z,12Z)-octadeca-9,12-dienyl, (9Z,12Z,15Z)-octadeca-9,12,15-trienyl, (Z)-icos-11-enyl, (11Z,14Z)-icosa-11,14-dienyl, 3,7,11-trimethyldodeca-2,6,10-trienyl, 3,7,11,15-tetramethylhexadec-2-enyl, (Z)-docos-13-enyl, and the like, and preferred examples thereof include (Z)-hexadec-9-enyl, (Z)-octadec-6-enyl, (Z)-octadec-9-enyl, (9Z,12Z)-octadeca-9,12-dienyl, (Z)-icos-11-enyl, (11Z,14Z)-icosa-11,14-dienyl, (Z)-docos-13-enyl, and the like.

The linear or branched alkynyl having 8 to 24 carbon atoms may be linear or branched alkynyl having 8 to 24 carbon atoms and having 1 to 3 triple bonds. Examples thereof include dodec-11-ynyl, tetradec-6-ynyl, hexadec-7-ynyl, hexadeca-5,7-diynyl, octadec-9-ynyl, and the like.

The alkyl moiety in the alkoxyethylene and the alkoxypropylene has the same definition as the linear or branched alkyl having 8 to 24 carbon atoms described above.

The alkenyl moiety in the alkenyloxyethylene and the alkenyloxypropylene has the same definition as the linear or branched alkenyl having 8 to 24 carbon atoms described above.

The alkynyl moiety in the alkynyloxyethylene and the alkynyloxypropylene has the same definition as the linear or branched alkynyl having 8 to 24 carbon atoms described above.

Incidentally, it is more preferred that R¹ and R² are the same and are each linear or branched alkyl, alkenyl, or alkynyl each having 8 to 24 carbon atoms. Further, in any case, R¹ and R² are each more preferably linear or branched alkyl or alkenyl, each having 8 to 24 carbon atoms, and further more preferably alkenyl.

It is preferred that R¹ and R² are the same or different and are each tetradecyl, hexadecyl, (Z)-tetradec-9-enyl, (Z)-hexadec-9-enyl, (Z)-octadec-6-enyl, (Z)-octadec-9-enyl, (E)-octadec-9-enyl, (Z)-octadec-11-enyl, (9Z,12Z)-octadeca-9,12-dienyl, (9Z,12Z,15Z)-octadeca-9,12,15-trienyl, (Z)-icos-11-enyl, (11Z,14Z)-icosa-11,14-dienyl, or (Z)-docos-13-enyl, it is more preferred that R¹ and R² are the same or different and are each hexadecyl, (Z)-hexadec-9-enyl, (Z)-octadec-6-enyl, (Z)-octadec-9-enyl, (9Z,12Z)-octadeca-9,12-dienyl, (Z)-icos-11-enyl, or (11Z,14Z)-icosa-11,14-dienyl, it is further more preferred that R¹ and R² are the same or different and are each (Z)-octadec-9-enyl or (9Z,12Z)-octadeca-9,12-dienyl, and it is most preferred that R¹ and R² are the same and are each (Z)-octadec-9-enyl or (9Z,12Z)-octadeca-9,12-dienyl.

It is also one of the preferred embodiments of the present invention that R¹ is linear or branched alkyl, alkenyl, or alkynyl, each having 16 to 24 carbon atoms, and R² is linear or branched alkyl, alkenyl, or alkynyl, each having 8 to 12 carbon atoms. In this case, it is more preferred that R¹ is linear alkenyl having 16 to 24 carbon atoms, and R² is linear alkyl having 8 to 12 carbon atoms, and it is most preferred that R¹ is (Z)-octadec-9-enyl or (9Z,12Z)-octadeca-9,12-dienyl, and R² is octyl, decyl, or dodecyl.

Examples of the alkyl having 1 to 3 carbon atoms represented by R³ and R⁴ include methyl, ethyl, propyl, isopropyl, and cyclopropyl, preferred examples thereof include methyl and ethyl, and more preferred examples thereof include methyl.

Examples of the alkylene having 2 to 6 carbon atoms represented by R³ and R⁴ include ethylene, propylene, butylene, pentylene, hexylene, and the like.

Examples of the alkyl having 1 to 6 carbon atoms represented by R⁵ include methyl, ethyl, propyl, isopropyl, cyclopropyl, butyl, isobutyl, sec-butyl, tert-butyl, cyclobutyl, cyclopropylmethyl, pentyl, isopentyl, sec-pentyl, neopentyl, tert-pentyl, cyclopentyl, hexyl, cyclohexyl, and the like, preferred examples thereof include methyl, ethyl, propyl, isopropyl, butyl, isobutyl, sec-butyl, tert-butyl, pentyl, isopentyl, sec-pentyl, tert-pentyl, neopentyl, hexyl, and the like, and more preferred examples thereof include methyl, ethyl, propyl, and the like.

Examples of the alkeny having 3 to 6 carbon atoms represented by R⁵ include allyl, 1-propenyl, butenyl, pentenyl, hexenyl, and the like, and preferred examples thereof include allyl and the like.

The monoalkylamino represented by R⁵ may be amino substituted with one substituent which is alkyl having 1 to 6 carbon atoms (having the same definition as described above), and examples thereof include methylamino, ethylamino, propylamino, butylamino, pentylamino, hexylamino, and the like, and preferred examples thereof include methylamino, ethylamino, and the like.

The amino and the monoalkylamino represented by R⁵ may form ammonio and monoalkylammonio, respectively, through coordination of a hydrogen ion with a lone pair on the nitrogen atom. The amino and the monoalkylamino include ammonic and monoalkylammonio, respectively.

In the present invention, the ammonio and the monoalkylammonio, in each of which a hydrogen ion is coordinated with a lone pair on the nitrogen atom of each of the amino and the monoalkylamino, may form a salt together with a pharmaceutically acceptable anion.

The alkoxy represented by R⁵ may be hydroxy substituted with alkyl having 1 to 6 carbon atoms (having the same definition as described above), and examples thereof include methoxy, ethoxy, propyloxy, butyloxy, pentyloxy, hexyloxy, and the like, and preferred examples thereof include methoxy, ethoxy, and the like.

The monoalkylcarbamoyl and the dialkylcarbamoyl represented by R⁵ may be carbamoyl substituted with one substituent and the same or different two substituents, respectively, wherein the substituent is alkyl having 1 to 6 carbon atoms (having the same definition as described above), and examples thereof include methylcarbamoyl, ethylcarbamoyl, propylcarbamoyl, butylcarbamoyl, pentylcarbamoyl, hexylcarbamoyl, dimethylcarbamoyl, diethylcarbamoyl, ethylmethylcarbamoyl, methylpropyl carbamoyl, butylmethylcarbamoyl, methylpentylcarbamoyl, hexylmethylcarbamoyl, and the like, and preferred examples thereof include methylcarbamoyl, ethylcarbamoyl, dimethylcarbamoyl, and the like.

Examples of the alkylene having 2 to 6 carbon atoms represented by R⁵ include ethylene, propylene, butylene, pentylene, hexylene, and the like.

Examples of the alkylene having 1 to 6 carbon atoms represented by X and Y include methylene, ethylene, propylene, butylene, pentylene, hexylene, and the like.

Further, in formula (I), X is preferably alkylene having 1 to 3 carbon atoms, and most preferably methylene or ethylene. In any case, it is preferred that Ry is a hydrogen atom, methyl, amino, methylamino, hydroxy, methoxy, or methyl substituted with 1 to 3 substituents selected from amino and hydroxy, or is combined together with R³ to form alkylene having 2 to 4 carbon atoms, and it is most preferred that R⁵ is a hydrogen atom or methyl, or is combined together with R³ to form propylene or butylene.

Each of the oxygen atoms in formula (I) may be replaced with a sulfur atom.

Compound (Ia) represented by formula (Ia) in which an oxygen atom in formula (I) is replaced with a sulfur atom:

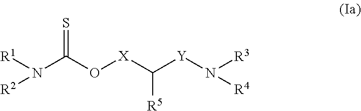

(wherein R¹, R², R³, R⁴, R⁵, X, and Y have the same definitions as described above, respectively) can be obtained by allowing a 1,3,2,4-dithiadiphosphetane 2,4-disulfide derivative such as Lawesson's Reagent (2,4-bis(4-methoxyphenyl)-1,3,2,4-dithiadiphosphetane 2,4-disulfide) to act on corresponding Compound (I).

The cationic lipid of the present invention may form a salt with a pharmaceutically acceptable anion.

In the present invention, examples of the pharmaceutically acceptable anion include inorganic ions such as a chloride ion, a bromide ion, a nitrate ion, a sulfate ion, and a phosphate ion, organic acid ions such as an acetate ion, an oxalate ion, a maleate ion, a fumarate ion, a citrate ion, a benzoate ion, and a methanesulfonate ion, and the like.

Next, a production method of Compound (I) will be described. Incidentally, in the following production method, in the case where a defined group changes under the conditions for the production method or is not suitable for carrying out the production method, the target compound can be produced by adopting the introduction and removal method of a protective group commonly used in synthetic organic chemistry (for example, the method described in Protective Groups in Organic Synthesis, Third Edition, written by T. W. Greene, John Wiley & Sons, Inc. (1999), or the like] or the like. In addition, if desired, the order of reaction steps such as introduction of a substituent can be altered.

### Production Method

Compound (I) can be produced by the following method.

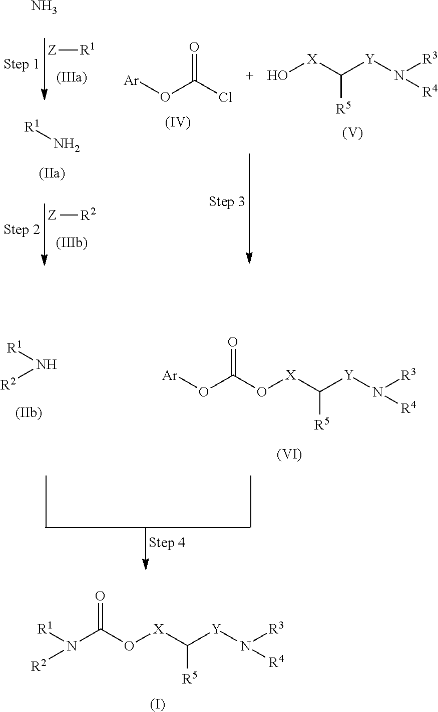

(wherein R¹, R², R³, R⁴, R⁵, X, and Y have the same definitions as described above, respectively, Z represents a leaving group, such as a chlorine atom, a bromine atom, an iodine atom, trifluoromethanesulfonyloxy, methanesulfonyloxy, benzenesulfonyloxy, or p-toluenesulfonyloxy, and Ar represents a substituted phenyl group such as p-nitrophenyl, o-nitrophenyl, or p-chlorophenyl, or an unsubstituted phenyl group.

### Steps 1 and 2

Compound (IIa) can be produced by treating ammonia and Compound (IIIa) without a solvent or in a solvent, if necessary, preferably in the presence of 1 to 10 equivalents of a base at a temperature between room temperature and 200° C. for 5 minutes to 100 hours. Further, Compound (IIb) can be produced by treating Compound (IIa) and Compound (IIIb) without a solvent or in a solvent, if necessary, preferably in the presence of 1 to 10 equivalents of a base at a temperature between room temperature and 200° C. for 5 minutes to 100 hours, followed by isolation.

Examples of the solvent include methanol, ethanol, dichloromethane, chloroform, 1,2-dichloroethane, toluene, ethyl acetate, acetonitrile, diethyl ether, tetrahydrofuran, 1,2-dimethoxyethane, dioxane, N,N-dimethylformamide, N,N-dimethylacetamide, N-methylpyrrolidone, pyridine, water, and the like. These are used alone or as a mixture.

Examples of the base include potassium carbonate, potassium hydroxide, sodium hydroxide, sodium methoxide, potassium tert-butoxide, triethylamine, diisopropylethylamine, N-methylmorpholine, pyridine, 1,3-diazabicyclo[5.4.0]-7-undecene (DBU), and the like.

Compound (IIIa) and Compound (IIib) can be obtained as commercially available products or by known methods (for example, “Dai 5-han Jikken Kagaku Kouza 13, Synthesis of Organic Compounds I”, 5th Ed., p. 374, Maruzen (2005)) or modified methods thereof.

Compound (IIb) in the case where R¹ and R² are the same can be obtained by using 2 equivalents or more of Compound (IIIa) in Step 1.

### Step 3

Compound (VI) can be produced by reacting Compound (IV) with Compound (V) without a solvent or in a solvent, if necessary, preferably in the presence of 1 to 10 equivalents of an additive, and/or if necessary, preferably in the presence of 1 to 10 equivalents of a base at a temperature between −20° C. and 150° C. for 5 minutes to 72 hours.

Examples of the solvent include dichloromethane, chloroform, 1,2-dichloroethane, toluene, ethyl acetate, acetonitrile, diethyl ether, tetrahydrofuran, 1,2-dimethoxyethane, dioxane, N,N-dimethylformamide, N,N-dimethylacetamide, N-methylpyrrolidone, dimethylsulfoxide, and the like. These can be used alone or as a mixture.

Examples of the additive include 1-hydroxybenzotriazole, 4-dimethylaminopyridine, and the like.

Examples of the base include the same bases as those described with respect to Steps 1 and 2.

Compound (IV) can be obtained as a commercially available product.

Compound (V) can be obtained as a commercially available product or by known methods (for example, “Dai 5-han, Jikken Kagaku Kouza 14, Synthesis of Organic Compounds II”, 5th Ed., p. 1, Maruzen (2005)) or modified methods thereof.

### Step 4

Compound (I) can be produced by reacting Compound (IIb) with Compound (VI) without a solvent or in a solvent, if necessary, preferably in the presence of 1 to 10 equivalents of an additive, and/or if necessary, preferably in the presence of 1 to 10 equivalents of a base at a temperature between −20° C. and 150° C. for 5 minutes to 72 hours.

Examples of the solvent and the additive include the same solvents and additives as those described with respect to Step 3.

Examples of the base include the same bases as those described with respect to Steps 1 and 2.

Compounds (Ia) to (Ic) represented by formulae (Ia) to (Ic) in which an oxygen atom in formula (I) is replaced with a sulfur atom:

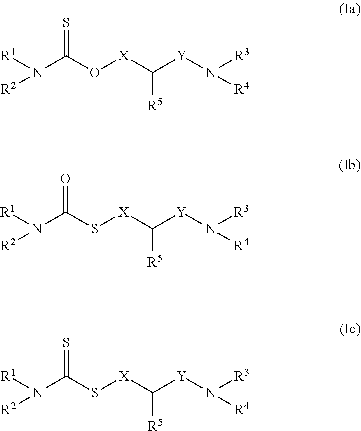

(wherein R¹, R², R³, R⁴, R⁵, X, and Y have the same definitions as described above, respectively) can be obtained by using Compounds (VIa) to (VIc) represented by formulae (VIa) to (VIc) in which an oxygen atom in formula (VI) is replaced with a sulfur atom:

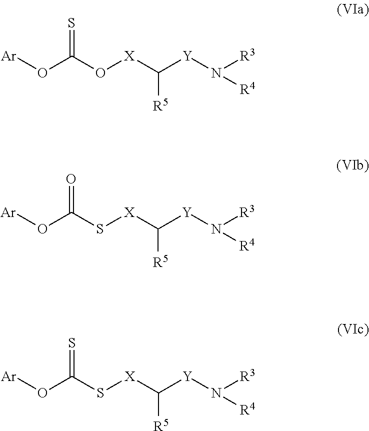

(wherein R³, R⁴, R⁵, X, Y, and Ar have the same definitions as described above, respectively) in Step 4.

The intermediates and the target compounds in the above-described respective production methods can be isolated and purified by separation and purification methods commonly used in synthetic organic chemistry, for example, filtration, extraction, washing, drying, concentration, recrystallization, various chromatography, and the like. In addition, it is also possible to subject the intermediate to the subsequent reaction without particularly purifying it.

In Compound (I), a hydrogen ion may be coordinated with a lone pair on the nitrogen atom in the structure, and the nitrogen atom may form a salt together with a pharmaceutically acceptable anion (having the same definition as described above). Compound (I) also includes compounds in which a hydrogen ion is coordinated with a lone pair on the nitrogen atom.

Among Compounds (I), there may exist compounds in the form of stereoisomers such as geometric isomers, optical isomers and the like, tautomers, and the like. Compound (I) includes all the possible isomers and mixtures thereof inclusive of these stereoisomers and tautomers.

Part or all of the respective atoms in Compound (I) may be replaced with a corresponding isotope atom. Compound (I) also includes such compounds in which part or all of the respective atoms are replaced with a corresponding isotope atom. For example, part or all of the hydrogen atoms in Compound (I) may be a hydrogen atom having an atomic weight of 2 (deuterium atom).

The compounds in which part or all of the respective atoms in Compound (I) are replaced with a corresponding isotope atom can be produced by the same methods as the above-described respective production methods using a commercially available building block. Further, the compounds in which part or all of the hydrogen atoms in Compound (I) are replaced with a deuterium atom can also be synthesized by using, for example, 1) a method in which a carboxylic acid or the like is deuterated by using deuterium peroxide under a basic condition (see U.S. Pat. No. 3,849,458), 2) a method in which an alcohol, a carboxylic acid, or the like is deuterated by using an iridium complex as a catalyst and using heavy water as a deuterium source (see Journal of American Chemical Society (J. Am. Chem. Soc.), Vol. 124, No. 10, 2092 (2002)), 3) a method in which a fatty acid is deuterated by using palladium-carbon as a catalyst and using only deuterium gas as a deuterium source (see LIPIDS, Vol. 9, No. 11, 913 (1974)), 4) a method in which acrylic acid, methyl acrylate, methacrylic acid, methyl methacrylate, or the like is deuterated by using a metal such as platinum, palladium, rhodium, ruthenium, or iridium as a catalyst and using heavy water or heavy water and deuterium gas as a deuterium source (see Japanese Published Examined Patent Application No. Hei 5-19536, and Japanese Published Unexamined Patent Application No. Shou 61-27764 and No. Shou 61-275241), 5) a method in which acrylic acid, methyl methacrylate, or the like is deuterated by using a catalyst such as palladium, nickel, copper, or copper chromite and using heavy water as a deuterium source (see Japanese Published Unexamined Patent Application No. Shou 63-19638), or the like.

Specific examples of Compound (I) obtained according to the present invention are shown in Tables 1 and 2. It should be noted, however, that the compounds of the present invention are not limited to these.

TABLE 1

| Compound | |
|---|---|
| No. | Structural formula |
| I-1 | 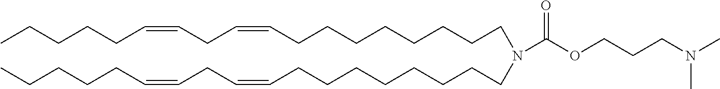 [SMILES: CCCCC/C=C\C/C=C\CCCCCCCCN(CCCCCCCC/C=C\C/C=C\CCCCC)C(=O)OCCCN(C)C] |
| I-2 | 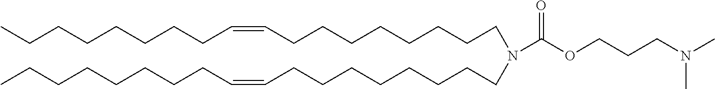 [SMILES: CCCCCCCC/C=C\CCCCCCCCN(CCCCCCCC/C=C\CCCCCCCC)C(=O)OCCCN(C)C] |
| I-3 | 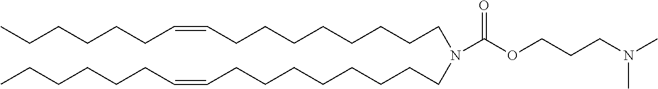 [SMILES: CCCCCC/C=C\CCCCCCCCN(CCCCCCCC/C=C\CCCCCC)C(=O)OCCCN(C)C] |
| I-4 | 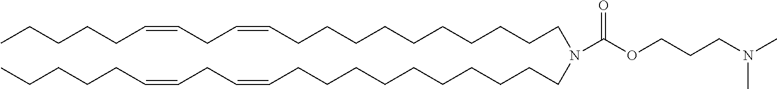 [SMILES: CCCCC/C=C\C/C=C\CCCCCCCCCCN(CCCCCCCCCC/C=C\C/C=C\CCCCC)C(=O)OCCCN(C)C] |
| I-5 | 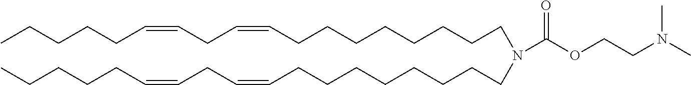 [SMILES: CCCCC/C=C\C/C=C\CCCCCCCCN(CCCCCCCC/C=C\C/C=C\CCCCC)C(=O)OCCN(C)C] |

TABLE 2

| Compound | |
|---|---|
| No. | Structural formula |
| I-6 | 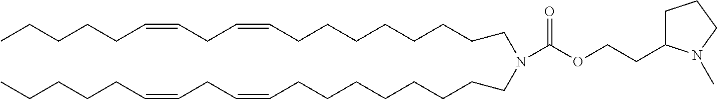 [SMILES: CCCCC/C=C\C/C=C\CCCCCCCCN(CCCCCCCC/C=C\C/C=C\CCCCC)C(=O)OCCC1CCCN1C] |
| I-7 | 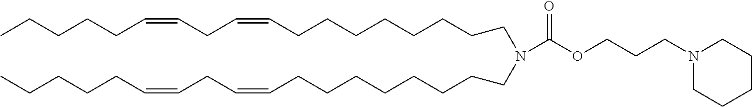 [SMILES: CCCCC/C=C\C/C=C\CCCCCCCCN(CCCCCCCC/C=C\C/C=C\CCCCC)C(=O)OCCCN1CCCCC1] |
| I-8 | 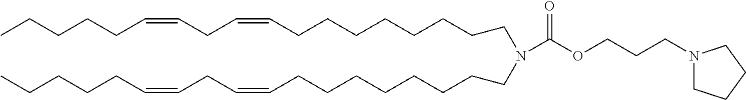 [SMILES: CCCCC/C=C\C/C=C\CCCCCCCCN(CCCCCCCC/C=C\C/C=C\CCCCC)C(=O)OCCCN1CCCC1] |
| I-9 | 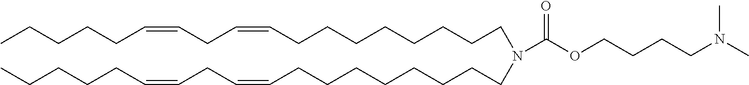 [SMILES: CCCCC/C=C\C/C=C\CCCCCCCCN(CCCCCCCC/C=C\C/C=C\CCCCC)C(=O)OCCCCN(C)C] |
| I-10 | 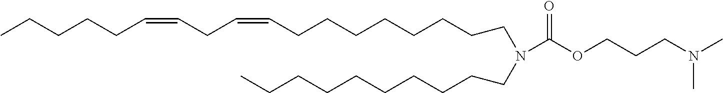 [SMILES: CCCCC/C=C\C/C=C\CCCCCCCCN(CCCCCCCCCC)C(=O)OCCCN(C)C] |
| I-11 | 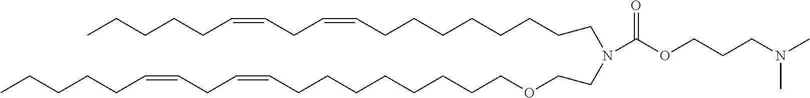 [SMILES: CCCCC/C=C\C/C=C\CCCCCCCCOCCN(CCCCCCCC/C=C\C/C=C\CCCCC)C(=O)OCCCN(C)C] |

The nucleic acid to be used in the present invention may be any molecule as long as it is a molecule obtained by polymerization of a nucleotide and/or a molecule having a function equivalent to that of the nucleotide. Examples thereof include RNA which is a polymer of a ribonucleotide, DNA which is a polymer of a deoxyribonucleotide, a chimera nucleic acid composed of RNA and DNA, and a nucleotide polymer in which at least one nucleotide of these nucleic acids is substituted with a molecule having a function equivalent to that of the nucleotide. In addition, a derivative containing at least one molecule obtained by polymerization of a nucleotide and/or a molecule having a function equivalent to that of the nucleotide is also included in the nucleic acid of the present invention. Incidentally, in the present invention, uridine U in RNA and thymine T in DNA can be replaced with each other.

Examples of the molecule having a function equivalent to that of a nucleotide include nucleotide derivatives and the like.

The nucleotide derivative may be any molecule as long as it is a molecule obtained by modifying a nucleotide. For example, for the purpose of enhancing the nuclease resistance or achieving stabilization against other decomposing factors as compared with RNA or DNA, increasing the affinity for a complementary strand nucleic acid, increasing the cellular permeability, or achieving the visualization, a molecule obtained by modifying a ribonucleotide or a deoxyribonucleotide, or the like is preferably used.

Examples of the nucleotide derivative include a sugar moiety-modified nucleotide, a phosphodiester bond-modified nucleotide, a base-modified nucleotide, and the like.

The sugar moiety-modified nucleotide may be any as long as it is a nucleotide in which part or all of the chemical structure of the sugar moiety of the nucleotide is modified or substituted with an arbitrary substituent or substituted with an arbitrary atom, however, a 2′-modified nucleotide is preferably used.

Examples of the modifying group in the sugar moiety-modified nucleotide include 2′-cyano, 2′-alkyl, 2′-substituted alkyl, 2′-alkenyl, 2′-substituted alkenyl, 2′-halogen, 2′-O-cyano, 2′-O-alkyl, 2′-O-substituted alkyl, 2′-O-alkenyl, 2′-O-substituted alkenyl, 2′-S-alkyl, 2′-S-substituted alkyl, 2′-S-alkenyl, 2′-S-substituted alkenyl, 2′-amino, 2′-NH-alkyl, 2′-NH-substituted alkyl, 2′-NH-alkenyl, 2′-NH-substituted alkenyl, 2′-SO-alkyl, 2′-SO-substituted alkyl, 2′-carboxy, 2′-CO-alkyl, 2′-CO-substituted alkyl, 2′-Se-alkyl, 2′-Se-substituted alkyl, 2′-SiH₂-alkyl, 2′-SiH₂-substituted alkyl, 2′-ONO₂, 2′-NO₂, 2′-N₃, a 2′-amino acid residue (a residue obtained by removing the hydroxyl group from the carboxylic acid of an amino acid), and a 2′-O-amino acid residue (having the same definition as the above-described amino acid residue). Further, additional examples thereof include a peptide nucleic acid (PNA) [Acc. Chem. Res., 32, 624 (1999)], an oxy-peptide nucleic acid (OPNA) [J. Am. Chem. Soc., 123, 4653 (2001)], a peptide ribonucleic acid (PRNA) [J. Am. Chem. Soc., 122, 6900 (2000)], and the like. Further, a ribose substituted with a modifying group at the 2′ position in the present invention also includes bridged nucleic acids (BNAs) having a structure in which the modifying group at the 2′ position is bridged to the carbon atom at the 4′ position, more specifically, locked nucleic acids (LNAs) in which the oxygen atom at the 2′ position is bridged to the carbon atom at the 4′ position via methylene, ethylene bridged nucleic acids (ENAs) [Nucleic Acid Research, 32, e175 (2004)], and the like.

As the modifying group in the sugar moiety-modified nucleotide, 2′-cyano, 2′-halogen, 2′-O-cyano, 2′-alkyl, 2′-substituted alkyl, 2′-O-alkyl, 2′-O-substituted alkyl, 2′-O-alkenyl, 2′-O-substituted alkenyl, 2′-Se-alkyl, and 2′-Se-substituted alkyl are preferred, 2′-cyano, 2′-fluoro, 2′-chloro, 2′-bromo, 2′-trifluoromethyl, 2′-O-methyl, 2′-O-ethyl, 2′-O-isopropyl, 2′-O-trifluoromethyl, 2′-O-[2-(methoxy)ethyl, 2′-O-(3-aminopropyl), 2′-O-(2-(N,N-dimethyl)aminooxy]ethyl, 2′-(3-(N,N-dimethylamino)propyl), 2′-O-2-{2-(N,N-dimethylamino)ethoxy ethyl}, 2′-O-[2-(methylamino)-2-oxoethyl], 2′-Se-methyl, and the like are more preferred, 2′-O-methyl, 2′-O-ethyl, 2′-fluoro, and the like are further more preferred, and 2′-O-methyl and 2′-O-ethyl are most preferred.

Further, the preferred range of the modifying group in the sugar moiety-modified nucleotide can also be defined based on its size, and a modifying group with a size corresponding to the size of fluoro to the size of —O-butyl is preferred, and a modifying group with a size corresponding to the size of —O-methyl to the size of —O-ethyl is more preferred.

The alkyl in the modifying group in the sugar moiety-modified nucleotide has the same definition as the alkyl having 1 to 3 carbon atoms in the cationic lipid of the present invention.

Examples of the alkenyl in the modifying group in the sugar moiety-modified nucleotide include alkenyl having 3 to 6 carbon atoms, for example, allyl, 1-propenyl, butenyl, pentenyl, hexenyl, and the like.

Examples of the halogen in the modifying group in the sugar moiety-modified nucleotide include a fluorine atom, a chlorine atom, a bromine atom, and an iodine atom.

Examples of the amino acid in the amino acid residue include aliphatic amino acids (specifically, glycine, alanine, valine, leucine, isoleucine, and the like), hydroxy amino acids (specifically, serine, thzeonine, and the like), acidic amino acids (specifically, aspartic acid, glutamic acid, and the like), acidic amino acid amides (specifically, asparagine, glutamine, and the like), basic amino acids (specifically, lysine, hydroxylysine, arginine, ornithine, and the like), sulfur-containing amino acids (specifically, cysteine, cystine, methionine, and the like), imino acids (specifically, proline, 4-hydroxy proline, and the like), and the like.

Examples of the substituent in the substituted alkyl and the substituted alkenyl in the modifying group in the sugar moiety-modified nucleotide include halogen (having the same definition as described above), hydroxy, sulfanyl, amino, oxo, —O-alkyl (the alkyl moiety of the —O-alkyl has the same definition as that of the above-describedalkyl), —S-alkyl (the alkyl moiety of the —S-alkyl has the same definition as that of the above-described alkyl), —NH-alkyl (the alkyl moiety of the —NH-alkyl has the same definition as that of the above-described alkyl), dialkylaminooxy (the two alkyl moieties of the dialkylaminooxy may be the same or different, and have the same definition as that of the above-described alkyl), dialkylamino (the two alkyl moieties of the dialkylamino may be the same or different, and have the same definition as that of the above-described alkyl), dialkylaminoalkyleneoxy (the two alkyl moieties of the dialkylaminoalkyleneoxy may be the same or different, and have the same definition as that of the above-described alkyl, and the alkylene moiety means a group obtained by removing one hydrogen atom from the above-described alkyl), and the like, and the number of substituents is preferably 1 to 3.

The phosphodiester bond-modified nucleotide may be any as long as it is a nucleotide in which part or all of the chemical structure of the phosphodiester bond of the nucleotide is modified or substituted with an arbitrary substituent or substituted with an arbitrary atom, and examples thereof include a nucleotide in which the phosphodiester bond is substituted with a phosphorothioate bond, a nucleotide in which the phosphodiester bond is substituted with a phosphorodithioate bond, a nucleotide in which the phosphodiester bond is substituted with an alkylphosphonate bond, a nucleotide in which the phosphodiester bond is substituted with a phosphoroamidate bond, and the like.

The base-modified nucleotide may be any as long as it is a nucleotide in which part or all of the chemical structure of the base of the nucleotide is modified or substituted with an arbitrary substituent or substituted with an arbitrary atom, and examples thereof include a nucleotide in which an oxygen atom in the base is substituted with a sulfur atom, a nucleotide in which a hydrogen atom is substituted with an alkyl group having 1 to 6 carbon atoms, a nucleotide in which a methyl group is substituted with a hydrogen atom or an alkyl group having 2 to 6 carbon atoms, and a nucleotide in which an amino group is protected by a protective group such as an alkyl group having 1 to 6 carbon atoms or an alkanoyl group having 1 to 6 carbon atoms.

Further, examples of the nucleotide derivative include those in which another chemical substance such as a lipid, a phospholipid, phenazine, folate, phenanthridine, anthraquinone, acridine, fluorescein, rhodamine, coumarin, or a pigment is added to a nucleotide or a nucleotide derivative in which at least one of the sugar moiety, the phosphodiester bond, and the base is modified. Specific examples thereof include 5′-polyamine-added nucleotide derivatives, cholesterol-added nucleotide derivatives, steroid-added nucleotide derivatives, bile acid-added nucleotide derivatives, vitamin-added nucleotide derivatives, Cy5-added nucleotide derivatives, Cy3-added nucleotide derivatives, 6-FAM-added nucleotide derivatives, biotin-added nucleotide derivatives, and the like.

In addition, the nucleotide derivative may form, together with another nucleotide or nucleotide derivative within the nucleic acid, a crosslinked structure such as an alkylene structure, a peptide structure, a nucleotide structure, an ether structure, an ester structure, or a structure combined with at least one of these structures.

Preferred examples of the nucleic acid to be used in the present invention include nucleic acids which suppress the expression of the target gene, and more preferred examples thereof include nucleic acids having an activity of suppressing the expression of the target gene by utilizing RNA interference (RNAi).

The target gene to be used in the present invention is not particularly limited as long as it is a gene which produces mRNA and is expressed, however, preferred examples thereof include genes associated with tumor or inflammation, for example, genes which encode proteins such as a vascular endothelial growth factor (hereinafter, abbreviated as “VEGF”), a vascular endothelial growth factor receptor (hereinafter, abbreviated as “VEGFR”), a fibroblast growth factor, a fibroblast growth factor receptor, a platelet-derived growth factor, a platelet-derived growth factor receptor, a liver cell growth factor, a liver cell growth factor receptor, a Kruppel-like factor (hereinafter, abbreviated as “KLF”), an Ets transcription factor, a nuclear factor, a hypoxia-inducible factor, a cell cycle-associated factor, a chromosomal duplication-associated factor, a chromosomal repair-associated factor, a microtubule-associated factor, a growth signaling pathway-associated factor, a growth-associated transcription factor, and an apoptosis-associated factor, and the like, and specific examples thereof include a VEGF gene, a VEGFR gene, a fibroblast growth factor gene, a fibroblast growth factor receptor gene, a platelet-derived growth factor gene, a platelet-derived growth factor receptor gene, a liver cell growth factor gene, a liver cell growth factor receptor gene, a KLF gene, an Ets transcription factor gene, a nuclear factor gene, a hypoxia-inducible factor gene, a cell cycle-associated factor gene, a chromosomal duplication-associated factor gene, a chromosomal repair-associated factor gene, a microtubule-associated factor gene (for example, a CKAP5 gene, etc.), a growth signaling pathway-associated factor gene (for example, a KRAS gene, etc.), a growth-associated transcription factor gene, an apoptosis-associated factor gene (for example, a BCL-2 gene, etc.), and the like.

As the target gene to be used in the present invention, a gene which is expressed in, for example, the liver, lung, kidney, or spleen is preferred, and a gene which is expressed in the liver is more preferred, and examples thereof include the above-described genes associated with tumor or inflammation, a hepatitis B virus genome, a hepatitis C virus genome, and genes which encode proteins such as an apolipoprotein (APO), hydroxymethyl glutaryl (HMG) CoA reductase, kexin type 9 serine protease (PCSK9), factor XII, a glucagon receptor, a glucocorticoid receptor, a leukotriene receptor, a thromboxane A2 receptor, a histamine H1 receptor, a carbonic anhydrase, an angiotensin converting enzyme, renin, p53, tyrosine phosphatase (PTP), a sodium-dependent glucose transport carrier, a tumor necrosis factor, and an interleukin, and the like.

As the nucleic acid which suppresses the expression of the target gene, any nucleic acid, for example, a double-stranded nucleic acid such as siRNA (short interference RNA) or miRNA (micro RNA), a single-stranded nucleic acid such as shRNA (short hairpin RNA), an antisense nucleic acid, or a ribozyme, or the like may be used as long as it is, for example, a nucleic acid which contains a base sequence complementary to a part of the base sequence of mRNA of a gene which encodes a protein or the like (target gene), and also suppresses the expression of the target gene, however, a double-stranded nucleic acid is preferred.

A nucleic acid which contains a base sequence complementary to a part of the base sequence of mRNA of the target gene is referred to as an antisense strand nucleic acid, and a nucleic acid which contains a base sequence complementary to the base sequence of the antisense strand nucleic acid is also referred to as a sense strand nucleic acid. The sense strand nucleic acid refers to a nucleic acid which can form a double-stranded forming region by pairing with the antisense strand nucleic acid such as a nucleic acid itself which is composed of a part of the base sequence of the target gene.

The double-stranded nucleic acid refers to a nucleic acid which has a double-stranded forming region by pairing two single strands. The double-stranded forming region refers to a region where a double strand is formed by the base pairing of nucleotides or derivatives thereof which constitute a double-stranded nucleic acid. The base pairs which constitute the double-stranded forming region are typically 15 to 27 base pairs, preferably 15 to 25 base pairs, more preferably 15 to 23 base pairs, further more preferably 15 to 21 base pairs, and particularly preferably 15 to 19 base pairs.

As the antisense strand nucleic acid in the double-stranded forming region, a nucleic acid which is composed of a part of the sequence of the target gene mRNA, or a nucleic acid which is obtained by substitution, deletion, or addition of 1 to 3 bases, preferably 1 to 2 bases, more preferably 1 base in the nucleic acid and has an activity of suppressing the expression of the target protein is preferably used. The length of the single-stranded nucleic acid which constitutes a double-stranded nucleic acid is typically 15 to 30 bases, preferably 1.5 to 29 bases, more preferably 15 to 27 bases, further more preferably 15 to 25 bases, particularly preferably 17 to 23 bases, and most preferably 19 to 21 bases.

The nucleic acid in either or both of the antisense strand and the sense strand which constitutes a double-stranded nucleic acid may have an additional nucleic acid which does not form a double strand contiguous with the double-stranded forming region on the 3′ side or the 5′ side. Such a region which does not form a double strand is also referred to as a protrusion (overhang).

As the double-stranded nucleic acid having a protrusion, a double-stranded nucleic acid having a protrusion composed of 1 to 4 bases, typically 1 to 3 bases at the 3′ end or the 5′ end of at least one single strand is used, however, a double-stranded nucleic acid having a protrusion composed of 2 bases is preferably used, and a double-stranded nucleic acid having a protrusion composed of dTdT or UU is more preferably used. A protrusion may be present on only the antisense strand, only the sense strand, and both of the antisense strand and the sense strand, however, a double-stranded nucleic acid in which a protrusion is present on both of the antisense strand and the sense strand is preferably used.

In addition, a sequence which is contiguous with the double-stranded forming region and partially or completely matches with the target gene mRNA, or a sequence which is contiguous with the double-stranded forming region and matches with the base sequence of the complementary strand of the target gene mRNA may also be used. Further, as the nucleic acid which suppresses the expression of the target gene, for example, a nucleic acid molecule which forms the above-described double-stranded nucleic acid by the activity of a ribonuclease such as Dicer (WO2005/089287), a double-stranded nucleic acid which does not have a protrusion at the 3′ end or the 5′ end, or the like can also be used.

When the above-described double-stranded nucleic acid is siRNA, at least a sequence of bases at the positions 1 to 17 from the 5′ end side to the 3′ end side of the antisense strand is a base sequence complementary to a sequence composed of 1.7 consecutive bases of the target gene mRNA. Preferably, a sequence of bases at the positions 1 to 19 from the 5′ end side to the 3′ end side of the antisense strand is a base sequence complementary to a sequence composed of consecutive 19 bases of the target gene mRNA, or a sequence of bases at the positions 1 to 21 is a base sequence complementary to a sequence composed of 21 consecutive bases of the target gene mRNA, or a sequence of bases at the positions 1 to 25 is a base sequence complementary to a sequence composed of 25 consecutive bases of the target gene mRNA.

Further, when the nucleic acid to be used in the present invention is siRNA, preferably 10 to 70%, more preferably 15 to 60%, further more preferably 20 to 50% of the sugars in the nucleic acid are riboses substituted with a modifying group at the 2′ position. In the present invention, the ribose substituted with a modifying group at the 2′ position means a ribose in which the hydroxyl group at the 2′ position is substituted with a modifying group. The configuration may be the same as or different from the configuration of the hydroxyl group at the 2′ position of the ribose, however, it is preferred that the configuration is the same as the configuration of the hydroxyl group at the 2′ position of the ribose. The ribose substituted with a modifying group at the 2′ position is included in a 2′-modified nucleotide among the sugar moiety-modified nucleotides, and the modifying group in the ribose substituted with a modifying group at the 2′ position has the same definition as the modifying group in the 2′-modified nucleotide.

The nucleic acid to be used in the present invention includes derivatives in which an oxygen atom or the like contained in the phosphate moiety, the ester moiety, or the like in the structure of the nucleic acid is substituted with another atom, for example, a sulfur atom or the like.

Further, in the sugar which binds to the base at the 5′ end of each of the antisense strand and the sense strand, the hydroxyl group at the 5′ position may be modified with a phosphate group or the above-described modifying group, or a group which is converted into a phosphate group or the above-described modifying group by an in vivo nuclease or the like.

Further, in the sugar which binds to the base at the 3′ end of each of the antisense strand and the sense strand, the hydroxyl group at the 3′ position may be modified with a phosphate group or the above-described modifying group, or a group which is converted into a phosphate group or the above-described modifying group by an in vivo nuclease or the like.

The single-stranded nucleic acid may be any as long as it is a nucleic acid which has a sequence complementary to a sequence composed of consecutive 15 to 27 bases, preferably consecutive 15 to 25 bases, more preferably consecutive 1.5 to 23 bases, further more preferably consecutive 15 to 21 bases, and particularly preferably consecutive 15 to 19 bases of the target gene, or a nucleic acid which is obtained by substitution, deletion, or addition of 1 to 3 bases, preferably 1 to 2 bases, more preferably 1 base in the nucleic acid and has an activity of suppressing the expression of the target protein. The length of the single-stranded nucleic acid is preferably 15 to 30 bases, and a single-stranded nucleic acid composed of 15 to 29 bases, more preferably 15 to 27 bases, further more preferably 15 to 25 bases, and particularly preferably 15 to 23 bases is preferably used.

As the single-stranded nucleic acid, a single-stranded nucleic acid obtained by connecting the antisense strand and the sense strand, which constitute the above-described double-stranded nucleic acid, via a spacer sequence (spacer oligonucleotide) may be used. As the spacer oligonucleotide, a single-stranded nucleic acid molecule composed of 6 to 12 bases is preferred, and the sequence thereof on the 5′ end side is preferably a UU sequence. Examples of the spacer oligonucleotide include a nucleic acid composed of a UUCAAGAGA sequence. As for the connection order of the antisense strand and the sense strand connected via the spacer oligonucleotide, either strand may be located on the 5′ side. The single-stranded nucleic acid is preferably a single-stranded nucleic acid such as shRNA which has a double-stranded forming region with a stem-loop structure. The single-stranded nucleic acid such as shRNA is typically 50 to 70 bases long.

A nucleic acid, which has a length of 70 bases or less, preferably 50 bases or less, more preferably 30 bases or less, and is designed so that it forms the above-described single-stranded nucleic acid or double-stranded nucleic acid by the activity of a ribonuclease or the like, may be used.

Incidentally, the nucleic acid to be used in the present invention may be produced by using a known RNA or DNA synthesis method, and an RFNA or DNA modification method.

The composition of the present invention is a composition containing Compound (I) and a nucleic acid, and examples thereof include a composition containing a complex between Compound (I) and a nucleic acid or a complex between a combination of a neutral lipid and/or a polymer with Compound (I) and a nucleic acid, a composition containing the complex and a lipid membrane which encapsulates the complex, and the like. The lipid membrane may be a lipid monolayer membrane (lipid monomolecular membrane) or a lipid bilayer membrane (lipid bimolecular membrane). Incidentally, in the lipid membrane, Compound (I), a neutral lipid and/or a polymer may be incorporated. Further, in the complex and/or the lipid membrane, a cationic lipid other than Compound (I) may be incorporated.

Further, additional examples of the composition of the present invention include a composition containing a complex between a cationic lipid other than Compound (I) and a nucleic acid or a complex between a combination of a neutral lipid and/or a polymer with a cationic lipid other than Compound (I) and a nucleic acid, and a lipid membrane which encapsulates the complex, wherein Compound (I) is incorporated in the lipid membrane, and the like. The lipid membrane in this case may also be a lipid monolayer membrane (lipid monomolecular membrane) or a lipid bilayer membrane (lipid bimolecular membrane). Further, in the lipid membrane, a cationic lipid other than Compound (I), a neutral lipid and/or a polymer may be incorporated.

In the composition of the present invention, a composition containing a complex between Compound (I) and a nucleic acid; a composition containing a complex between Compound (I) and a nucleic acid, and a lipid membrane which encapsulates the complex, wherein Compound (I) is incorporated in the lipid membrane; and a composition containing a complex between a cationic lipid other than Compound (I) and a nucleic acid, and a lipid membrane which encapsulates the complex, wherein Compound (I) is incorporated in the lipid membrane are more preferred, a composition containing a complex between Compound (I) and a nucleic acid; and a composition containing a complex between Compound (I) and a nucleic acid, and a lipid membrane which encapsulates the complex, wherein Compound (I) is incorporated in the lipid membrane are further more preferred, and a composition containing a complex between Compound (I) and a nucleic acid, and a lipid membrane which encapsulates the complex, wherein Compound (I) is incorporated in the lipid membrane is most preferred. Incidentally, in any case, in the lipid membrane, a neutral lipid and/or a polymer may be incorporated. Further, in the complex and/or the lipid membrane, a cationic lipid other than Compound (I) may be incorporated.

Examples of the form of the complex include a complex between a nucleic acid and a membrane composed of a lipid monolayer (reversed micelle); a complex between a nucleic acid and a liposome; a complex between a nucleic acid and a micelle, and the like in any case, and preferred examples thereof include a complex between a nucleic acid and a membrane composed of a lipid monolayer; and a complex between a nucleic acid and a liposome.

Examples of the composition containing a complex and a lipid bilayer membrane which encapsulates the complex include a composition containing the complex and a liposome composed of a lipid bilayer membrane which encapsulates the complex and the like.

Incidentally, in the composition of the present invention, one or more types of Compounds (I) may be used, and further, a cationic lipid other than Compound (I) may be mixed with Compound (I).

Examples of the cationic lipid other than Compound (I) include DOTMA, DOTAP, and the like disclosed in Japanese Published Unexamined Patent Application No. Shou 61-161246 (U.S. Pat. No. 5,049,386), DORIE, DOSPA, and the like disclosed in WO91/016024 and WO97/019675, DLinDMA and the like disclosed in WO2005/121348, DLin-K-DMA disclosed in WO2009/086558, (3R,4R)-3,4-bis((Z)-hexadec-9-enyloxy)-1-methylpyrrolidine, N-methyl-N,N-bis-(2-((Z)-octadec-6-enyloxy)ethyl)amine, and the like disclosed in WO2011/136368, preferred examples thereof include cationic lipids having a tertiary amine moiety with two unsubstituted alkyl groups or a quaternary ammonium moiety with three unsubstituted alkyl groups such as DOTMA, DOTAP, DORIE, DOSPA, DLinDMA, and DLin-K-DMA, and more preferred examples thereof include cationic lipids having the tertiary amine moiety. It is more preferred that the unsubstituted alkyl group of the tertiary amine moiety and the quaternary ammonium moiety is a methyl group.

Incidentally, the composition of the present invention can contain a nucleic acid, however, it can also contain a compound chemically close to a nucleic acid.

The composition of the present invention can be produced by known production methods or modified methods thereof and may be a composition produced by any production method. For example, in the production of a composition containing a liposome as one of the compositions, a known liposome preparation method can be applied. Examples of the known liposome preparation method include a liposome preparation method by Bangham et al. (see “The Journal of Molecular Biology (J. Mol. Biol.)”, 1965, Vol. 13, pp. 238-252), an ethanol injection method (see “The Journal of Cell Biology (J. Cell. Biol.)”, 1975, Vol. 66, pp. 621-634), a French press method (see “The FEBS Letters (FEBS Lett.)”, 1979, Vol. 99, pp. 210-214), a freeze-thawing method (see “The Archives of Biochemistry and Biophysics (Arch. Biochem. Biophys.)”, 1981, Vol. 212, pp. 186-194), a reverse phase evaporation method (see “The Proceedings of the National Academy of Sciences of the United States of America (Proc. Natl. Acad. Sci. USA)”, 1978, Vol. 75, pp. 4194-4198), a pH gradient method (see, for example, Japanese Examined Patent Publications Nos. 2572554 and 2659136, etc.), and the like. As a solution for dispersing a liposome in the production of a liposome, for example, water, an acid, an alkali, any of a variety of buffer solutions, saline, an amino acid infusion, or the like can be used. In addition, in the production of a liposome, it is also possible to add, for example, an antioxidant such as citric acid, ascorbic acid, cysteine, or ethylenediaminetetraacetic acid (EDTA), an isotonic agent such as glycerin, glucose, or sodium chloride, or the like. In addition, a liposome can also be produced by, for example, dissolving a lipid or the like in an organic solvent such as ethanol, distilling off the solvent, adding saline or the like, followed by stirring the mixture by shaking, thereby forming a liposome.

Further, the composition of the present invention can be produced by, for example, a method in which Compound (I) or a mixture of Compound (I) and a cationic lipid other than Compound (I) is dissolved in chloroform in advance, and then, an aqueous solution of a nucleic acid and methanol are added thereto followed by mixing, thereby forming a cationic lipid/nucleic acid complex, and further, a chloroform layer is taken out, and a polyethylene glycolated phospholipid, a neutral lipid, and water are added thereto, thereby forming a water-in-oil (W/O) emulsion, and the formed emulsion is treated by a reverse phase evaporation method (see Japanese Patent Domestic Announcement No. 2002-508765), a method in which a nucleic acid is dissolved in an acidic aqueous electrolyte solution, a lipid (in ethanol) is added thereto, the concentration of ethanol is decreased to 20 v/v %, thereby preparing a liposome encapsulating the nucleic acid, followed by sizing filtration and dialysis to remove an excess amount of ethanol, and thereafter, the sample is further dialyzed while increasing the pH, thereby removing the nucleic acid adhering to the surface of the liposome (see Japanese Patent Domestic Announcement No. 2002-501511 and Biochimica et Biophysica Acta, 2001, Vol. 1510, pp. 1.52-166), or the like.

Among the compositions of the present invention, a composition containing a complex between Compound (I) and a nucleic acid, or a complex between a combination of a neutral lipid and/or a polymer with Compound (I) and a nucleic acid and a liposome containing a lipid bilayer membrane which encapsulates the complex can be produced according to the production method described in, for example, WO02/28367, WO2006/080118, or the like.

Further, among the compositions of the present invention, for example, a composition containing a complex between Compound (I) and a nucleic acid, or a complex between a combination of a neutral lipid and/or a polymer with Compound (I) and a nucleic acid, and a lipid membrane which encapsulates the complex, a composition containing a complex between a cationic lipid other than Compound (I) and a nucleic acid, or a complex between a combination of a neutral, lipid and/or a polymer with a cationic lipid other than Compound (I) and a nucleic acid, and a lipid membrane which encapsulates the complex, wherein Compound (I) is incorporated in the lipid membrane, or the like can be obtained by producing each complex according to the production method described in WO02/28367, WO2006/080118, or the like, dispersing the complex in water or an aqueous solution of 0 to 20% ethanol without dissolving the complex (A solution), and separately dissolving each lipid membrane component in, for example, an aqueous solution of ethanol (B solution), mixing A solution and B solution in equal amounts, and further adding water thereto appropriately. Incidentally, as the cationic lipid in A solution or B solution, one or more types of Compounds (I) or cationic lipids other than Compound (I) may be used, and further, Compound (I) and a cationic lipid other than Compound (I) may be mixed with each other and used in combination.

Incidentally, in the present invention, compositions in which during the production and after the production of the composition containing a complex between Compound (I) and a nucleic acid, or a complex between a combination of a neutral lipid and/or a polymer with Compound (I) and a nucleic acid, and a lipid membrane which encapsulates the complex, the composition containing a complex between a cationic lipid other than Compound (I) and a nucleic acid, or a complex between a combination of a neutral lipid and/or a polymer with a cationic lipid other than Compound (I) and a nucleic acid, and a lipid membrane which encapsulates the complex, wherein Compound (I) is incorporated in the lipid membrane, and the like, the structures of the complex and the membrane are displaced due to an electrostatic interaction between the nucleic acid in the complex and the cationic lipid in the lipid membrane or fusion between the cationic lipid in the complex and the cationic lipid in the lipid membrane are also included in the composition containing a complex between Compound (I) and a nucleic acid, or a complex between a combination of a neutral lipid and/or a polymer with Compound (I) and a nucleic acid, and a lipid membrane which encapsulates the complex, the composition containing a complex between a cationic lipid other than Compound (I) and a nucleic acid, or a complex between a combination of a neutral lipid and/or a polymer with a cationic lipid other than Compound (I) and a nucleic acid, and a lipid membrane which encapsulates the complex, wherein Compound (I) is incorporated in the lipid membrane, and the like, respectively.

In the composition of the present invention, the total number of molecules of Compound (I) in the complex is preferably 0.5 to 4 times, more preferably 1.5 to 3.5 times, further more preferably 2 to 3 times the number of phosphorus atoms in the nucleic acid. Further, the total number of molecules of Compound (I) and the cationic lipid other than Compound (I) in the complex is preferably 0.5 to 4 times, more preferably 1.5 to 3.5 times, further more preferably 2 to 3 times the number of phosphorus atoms in the nucleic acid.

In the composition of the present invention, the total number of molecules of Compound (I) in the composition containing a complex and a lipid membrane which encapsulates the complex is preferably 1 to 10 times, more preferably 2.5 to 9 times, further more preferably 3.5 to 8 times the number of phosphorus atoms in the nucleic acid. Further, the total number of molecules of Compound (I) and the cationic lipid other than Compound (I) in the composition is preferably 1 to 10 times, more preferably 2.5 to 9 times, further more preferably 3.5 to 8 times the number of phosphorus atoms in the nucleic acid.

The neutral lipid may be any lipid selected from a simple lipid, a complex lipid, and a derived lipid, and examples thereof include a phospholipid, a glyceroglycolipid, a sphingoglycolipid, a sphingoid, a sterol, and the like. However, the neutral lipid is not limited thereto.

When the composition of the present invention contains a neutral lipid, the total number of molecules of the neutral lipid is preferably 0.1 to 1.8 times, more preferably 0.3 to 1.1 times, further more preferably 0.4 to 0.9 times the total number of molecules of Compound (I) and the cationic lipid other than Compound (I). In any case, in the composition of the present invention, a neutral lipid may be contained in the complex, and also may be contained in the lipid membrane which encapsulates the complex, and it is more preferred that a neutral lipid is contained in at least the lipid membrane which encapsulates the complex, and it is further more preferred that a neutral lipid is contained in both of the complex and the lipid membrane which encapsulates the complex.

Examples of the phospholipid as the neutral lipid include natural and synthetic phospholipids such as phosphatidylcholines (specifically, soybean phosphatidylcholine, egg yolk phosphatidylcholine (EPC), distearoyl phosphatidylcholine (DSPC), dipalmitoyl phosphatidylcholine (DPPC), palmitoyloleoyl phosphatidylcholine (POPC), dimyristoyl phosphatidylcholine (DMPC), dioleoyl phosphatidylcholine (DOPC), and the like), phosphatidylethanolamines (specifically, distearoyl phosphatidylethanolamine (DSPE), dipalmitoyl phosphatidylethanolamine (DPPE), dioleoyl phosphatidylethanolamine (DOPE), dimyristoyl phosphoethanolamine (DMPE), 16-0-monomethyl PE, 16-0-dimethyl PE, 18-1-trans PE, palmitoyloleoyl-phcphosphatidylethanolamine (POPE), 1-stearoyl-2-oleoyl-phosphatidylethanolamine (SOPE), and the like), glycerophospholipids (specifically, phosphatidylserine, phosphatidic acid, phosphatidylglycerol, phosphatidylinositol, palmitoyloleoyl phosphatidylglycerol (POPG), lysophosphatidylcholine, and the like), sphingophospholipids (specifically, sphingomyelin, ceramide phosphoethanolamine, ceramide phosphoglycerol, ceramide phosphoglycerophosphate, and the like), glycerophosphonolipids, sphingophosphonolipids, natural lecithins (specifically, egg yolk lecithin, soybean lecithin, and the like), and hydrogenated phospholipids (specifically, hydrogenated soybean phosphatidylcholine, and the like), and the like.

Examples of the glyceroglycolipid as the neutral lipid include sulfoxyribosyl glyceride, diglycosyl diglyceride, digalactosyl diglyceride, galactosyl diglyceride, glycosyl diglyceride, and the like.

Examples of the sphingoglycolipid as the neutral lipid include galactosyl cerebroside, lactosyl cerebroside, ganglioside, and the like.

Examples of the sphingoid as the neutral lipid include sphingan, icosasphingan, sphingosine, a derivative thereof, and the like. Examples of the derivative include those in which —NH₂ of sphingan, icosasphingan, sphingosine, or the like is replaced with —NHCO(CH₂)xCH₃ (in the formula, x is an integer of 0 to 18, and is preferably 6, 12 or 18), and the like.

Examples of the sterol as the neutral lipid include cholesterol, dihydrocholesterol, lanosterol, β-sitosterol, campesterol, stigmasterol, brassicasterol, ergocasterol, fucosterol, 3β-[N—(N′,N′-dimethylaminoethyl)carbamoyl]cholesterol (DC-Chol), and the like.

Examples of the polymer include micelles composed of one or more members selected from a protein, albumin, dextran, polyfect, chitosan, dextran sulfate, a polymer such as poly-L-lysine, polyethyleneimine, polyaspartic acid, a styrene-maleic acid copolymer, an isopropylacrylamide-acrylpyrrolidone copolymer, a polyethylene glycol-modified dendrimer, polylactic acid, polylactic acid polyglycolic acid, or polyethylene glycolated polylactic acid, and a salt thereof.

Here, the salt of the polymer includes, for example, a metal salt, an ammonium salt, an acid addition salt, an organic amine addition salt, an amino acid addition salt, and the like. Examples of the metal, salt include alkali metal salts such as a lithium salt, a sodium salt, and a potassium salt, alkaline earth metal salts such as a magnesium salt and a calcium salt, an aluminum salt, a zinc salt, and the like. Examples of the ammonium salt include salts of ammonium, tetramethylammonium, and the like. Examples of the acid addition salt include inorganic acid salts such as a hydrochloride, a sulfate, a nitrate, and a phosphate, and organic acid salts such as an acetate, a maleate, a fumarate, and a citrate. Examples of the organic amine addition salt include addition salts of morpholine, piperidine, and the like. Examples of the amino acid addition salt include addition salts of glycine, phenylalanine, aspartic acid, glutamic acid, lysine, and the like.

Further, in any case, the composition of the present invention preferably contains, for example, a lipid derivative or a fatty acid derivative of at least one substance selected from a sugar, a peptide, a nucleic acid, and a water-soluble polymer, or a surfactant or the like. Such a member may be contained in the complex or in the lipid membrane which encapsulates the complex, and it is more preferred that such a member is contained in both of the complex and the lipid membrane which encapsulates the complex.

When the composition of the present invention contains a lipid derivative or a fatty acid derivative of at least one substance selected from a sugar, a peptide, a nucleic acid, and a water-soluble polymer, the total number of molecules of the lipid derivative and the fatty acid derivative of at least one substance selected from a sugar, a peptide, a nucleic acid, and a water-soluble polymer is preferably 0.05 to 0.3 times, more preferably 0.07 to 0.25 times, further more preferably 0.1 to 0.2 times the total number of molecules of Compound (I) and the cationic lipid other than Compound (I).

As the lipid derivative or the fatty acid derivative of at least one substance selected from a sugar, a peptide, a nucleic acid, and a water-soluble polymer, or the surfactant, preferred is a lipid derivative or a fatty acid derivative of a glycolipid or a water-soluble polymer, and more preferred is a lipid derivative or a fatty acid derivative of a water-soluble polymer. The lipid derivative or the fatty acid derivative of at least one substance selected from a sugar, a peptide, a nucleic acid, and a water-soluble polymer, or the surfactant is preferably a substance having dual properties as follows: a part of the molecule has a property of binding to another constituent component of the composition through, for example, hydrophobic affinity, electrostatic interaction, or the like, and another part of the molecule has a property of binding to a solvent when producing the composition through, for example, hydrophilic affinity, electrostatic interaction, or the like.

Examples of the lipid derivative or the fatty acid derivative of a sugar, a peptide, or a nucleic acid include those obtained by binding a sugar such as sucrose, sorbitol, or lactose, a peptide such as a casein-derived peptide, an egg white-derived peptide, a soybean-derived peptide, or glutathione, or a nucleic acid such as DNA, RNA, a plasmid, siRNA, or ODN to the neutral lipid as exemplified above in the definition of the composition or the cationic lipid of the present invention or a fatty acid such as stearic acid, palmitic acid, myristic acid, or lauric acid, and the like.

Further, the lipid derivative or the fatty acid derivative of a sugar also includes, for example, the glyceroglycolipids and the sphingoglycolipids as exemplified above in the definition of the composition, and the like.

Examples of the lipid derivative or the fatty acid derivative of a water-soluble polymer include those obtained by binding polyethylene glycol, polyglycerin, polyethyleneimine, polyvinyl alcohol, polyacrylic acid, polyacrylamide, an oligosaccharide, dextrin, water-soluble cellulose, dextran, chondroitin sulfate, polyglycerin, chitosan, polyvinylpyrrolidone, polyaspartic acid amide, poly-L-lysine, mannan, pullulan, oligoglycerol, or the like or a derivative thereof to the neutral lipid as exemplified above in the definition of the composition, the cationic lipid of the present invention, or a fatty acid such as stearic acid, palmitic acid, myristic acid, or lauric acid, salts thereof, and the like. More preferred examples thereof include lipid derivatives or fatty acid derivatives of a polyethylene glycol derivative, a polyglycerin derivative, or the like, and salts thereof. Further more preferred examples thereof include lipid derivatives or fatty acid derivatives of a polyethylene glycol derivative, and salts thereof.

Examples of the lipid derivatives or the fatty acid derivatives of a polyethylene glycol derivative include polyethylene glycolated lipids (specifically, polyethylene glycol-phosphatidylethanolamines (more specifically, 1,2-distearoyl-sn-glycero-3-phosphoethanolamine-N-[methoxy(polyethylene glyco)-20001 (PEG-DSPE), 1,2-dimyristoyl-sn-glycero-3-phosphoethanolamine-N-methoxy(polyethylene glycol)-2000](PEG-DMPE), and the like)), polyoxyethylene hydrogenated castor oil 60, CREMOPHOR EL, and the like), polyethylene glycol sorbitan fatty acid esters (specifically, polyoxyethylene sorbitan monooleate, and the like), and polyethylene glycol fatty acid esters, and the like, and more preferred examples thereof include polyethylene glycolated lipids.

Examples of the lipid derivatives or the fatty acid derivatives of a polyglycerin derivative include polyglycerolated lipids (specifically, polyglycerin-phosphatidylethanolamines and the like), polyglycerin fatty acid esters, and the like, and more preferred examples thereof include polyglycerolated lipids.

Examples of the surfactant include polyoxyethylene sorbitan monooleates (specifically, Polysorbate 80 and the like), polyoxyethylene polyoxypropylene glycols (specifically, Pluronic F68 and the like), sorbitan fatty acid esters (specifically, sorbitan monolaurate, sorbitan monooleate, and the like), polyoxyethylene derivatives (specifically, polyoxyethylene hydrogenated castor oil 60, polyoxyethylene lauryl alcohol, and the like), glycerin fatty acid esters, polyethylene glycol alkyl ethers, and the like, and preferred examples thereof include polyoxyethylene polyoxypropylene glycols, glycerin fatty acid esters, polyethylene glycol alkyl ethers, and the like.

Further, the complex and the lipid membrane in the composition of the present invention can also be arbitrarily subjected to surface modification with, for example, a polymer, a polyoxyethylene derivative, or the like [see Stealth Liposomes, edited by D. D. Lasic and F. Martin, CRC Press Inc., US, 1995, pp. 93-102]. Examples of the polymer which can be used for the surface modification include dextran, pullulan, mannan, amylopectin, hydroxyethyl starch, and the like. Examples of the polyoxyethylene derivative include Polysorbate 80, Pluronic F68, polyoxyethylene hydrogenated castor oil 60, polyoxyethylene lauryl alcohol, PEG-DSPE, and the like. By the surface modification, in the complex and the lipid membrane in the composition of the present invention, a lipid derivative or a fatty acid derivative of at least one substance selected from a sugar, a peptide, a nucleic acid, and a water-soluble polymer, or a surfactant can be incorporated.

Further, by the covalent binding of a targeting ligand to a polar head residue of the lipid component of each lipid nanoparticle of the present invention, the targeting ligand can also be arbitrarily bound directly to the surface of the lipid nanoparticle of the present invention (see WO2006/116107).

The average particle diameter of the complex or the lipid membrane which encapsulates the complex in the composition of the present invention may be freely selected as desired, but is preferably adjusted to the average particle diameter described below. Examples of a method for adjusting the average particle diameter include an extrusion method, a method in which a large multilamellar liposome (MLV) or the like is mechanically pulverized (specifically, using Manton-gaulin, a microfluidizer, or the like) (see “Emulsion and Nanosuspensions for the Formulation of Poorly Soluble Drugs”, written and edited by R. H. Muller, S. Benita, and B. Bohm, Scientific Publishers, Stuttgart, Germany, 1998, pp. 267-294), and the like.

As for the size of the complex or the lipid membrane which encapsulates the complex in the composition of the present invention, the average particle diameter thereof is preferably about 10 nm to 1000 nm, more preferably about 30 nm to 300 nm, and further more preferably about 50 nm to 200 nm.

By administering the composition of the present invention to a mammalian cell, the nucleic acid in the composition of the present invention can be introduced into the cell.

A method for administering the composition of the present invention to a mammalian cell in vivo may be carried out according to a known transfection procedure which can be carried out in vivo. For example, by intravenously administering the composition of the present invention to mammals including humans, the composition is delivered to, for example, an organ or a site affected by cancer or inflammation, and the nucleic acid in the composition of the present invention can be introduced into the cells in this organ or site where the composition has been delivered. The organ or the site affected by cancer or inflammation is not particularly limited, but examples thereof include stomach, large intestine, liver, lung, spleen, pancreas, kidney, bladder, skin, blood vessel, eye ball, and the like. In addition, by intravenously administering the composition of the present invention to mammals including humans, the composition can be delivered to, for example, the blood vessel, liver, lung, spleen, and/or kidney, and the nucleic acid in the composition of the present invention can be introduced into the cells in the organ or the site where the composition has been delivered. The cells in the liver, lung, spleen, and/or kidney may be any of normal cells, cells associated with cancer or inflammation, and cells associated with other diseases.

If the nucleic acid in the composition of the present invention is a nucleic acid having an activity of suppressing the expression of the target gene by utilizing RNA interference (RNAi), the nucleic acid or the like which suppresses the expression of the gene can be introduced to mammalian cells in vivo, and the expression of the gene or the like can be suppressed. The administration target is preferably a human.

In addition, if the target gene for the composition of the present invention is, for example, a gene associated with the expression in the liver, lung, kidney, or spleen, preferably a gene which is expressed in the liver, the composition of the present invention can be used as a therapeutic agent or a preventive agent for a disease associated with the liver, lung, kidney, or spleen, preferably a therapeutic agent or a preventive agent for a disease associated with the liver.

Namely, the present invention also provides a method for treating a disease associated with the liver, lung, kidney, or spleen, including administering the composition of the present invention described above to a mammal. The administration target is preferably a human, and more preferably a human suffering from a disease associated with the liver, lung, kidney, or spleen.

Further, the composition of the present invention can also be used as a tool for verifying the effectiveness of suppressing the target gene in an in vivo drug efficacy evaluation model with respect to a therapeutic agent or a preventive agent for a disease associated with the liver, lung, kidney, or spleen.

The composition of the present invention can also be used as a preparation for, for example, stabilizing the above-described nucleic acid in biological components such as blood components (for example, blood, gastrointestinal tract, or the like), reducing side effects, increasing the drug accumulation in a tissue or an organ including the expression site of the target gene, and so on.

When the composition of the present invention is used as a therapeutic agent or a preventive agent for a disease or the like associated with the liver, lung, kidney, or spleen, which is a pharmaceutical preparation, it is desirable to use an administration route which is most effective for the treatment. Examples thereof include parenteral administration and oral administration such as intraoral administration, intratracheal administration, intrarectal administration, subcutaneous administration, intramuscular administration, and intravenous administration, and preferred examples thereof include intravenous administration and intramuscular administration, and more preferred examples thereof include intravenous administration.

The dose varies depending on the disease conditions or age of the administration target, the administration route, or the like, however, for example, the composition may be administered at a daily dose of about 0.1 g to 1000 mg in terms of the nucleic acid.

Examples of a preparation suitable for the intravenous administration or intramuscular administration include an injection, and it is also possible to use a dispersion liquid of the composition prepared by the above-described method as it is in the form of, for example, an injection or the like. However, it can also be used after removing the solvent from the dispersion liquid by, for example, filtration, centrifugation, or the like, or after lyophilizing the dispersion liquid and/or after lyophilizing the dispersion liquid supplemented with an excipient such as mannitol, lactose, trehalose, maltose, or glycine.

In the case of an injection, it is preferred to prepare the injection by mixing, for example, water, an acid, an alkali, any of a variety of buffer solutions, saline, an amino acid infusion, or the like with the above-described dispersion liquid of the composition or the above-described composition obtained by removing the solvent or lyophilization. In addition, it is also possible to prepare the injection by adding an antioxidant such as citric acid, ascorbic acid, cysteine, or EDTA, an isotonic agent such as glycerin, glucose, or sodium chloride, or the like. Further, the injection can also be cryopreserved by adding a cryopreservative such as glycerin.

### EXAMPLES

Next, the present invention is specifically described with reference to Examples and Test Examples. However, the present invention is not limited to these Examples and Test Examples.

Incidentally, the proton nuclear magnetic resonance spectra (¹H NMR) shown in Examples and Reference Examples are those measured at 270 MHz, 300 MHz, 400 MHz, or 500 MHz, and there may be the case where an exchangeable proton is not clearly observed depending on the compound and the measurement conditions. Incidentally, as the expression for the multiplicity of a signal, a conventionally used expression is employed, however the symbol “br” indicates that the signal is an apparent broad signal.

### Reference Example 1

### Di((9Z,12Z)-octadeca-9,12-dienyl)amine (Compound IIb-1)

To ammonia (manufactured by Tokyo Chemical Industry Co., Ltd., about 2 mol/L methanol solution, 18.0 mL, 36.0 mmol), (9Z,12Z)-octadeca-9,12-dienyl methanesulfonate (manufactured by Nu-Chek Prep, Inc., 1.55 g, 4.50 mol) was added, followed by heating and stirring at 130° C. for 3 hours using a microwave reactor. To the reaction mixture, a saturated sodium bicarbonate solution was added, and the mixture was extracted 5 times with chloroform. The organic layers were combined, washed with saturated brine, and dried over anhydrous magnesium sulfate. Thereafter, the resultant was filtered and concentrated under reduced pressure, whereby a crude product of (9Z,12Z)-octadeca-9,12-dienylamine was obtained.

To the obtained crude product, (9Z,12Z)-octadeca-9,12-dienyl methanesulfonate (manufactured by Nu-Chek Prep, Inc., 1.24 g, 3.60 mmol) and a 50% sodium hydroxide aqueous solution (1.44 g, 18.0 mmol) were added, followed by heating and stirring at 110° C. for 60 minutes in an oil bath. After cooling to room temperature, the reaction mixture was diluted with ethyl acetate, washed successively with water and saturated brine, and dried over anhydrous magnesium sulfate. Thereafter, the resultant was filtered and concentrated under reduced pressure. The obtained residue was purified by silica gel column chromatography (chloroform/methanol=100/0 to 95/5), whereby Compound IIb-1 (0.838 g, 36.2%) was obtained.

ESI-MS m/z: 515 (M+H)⁺; ¹H-NMR (CDCl₃) δ: 0.89 (t, J=6.9 Hz, 6H), 1.30 (br s, 33H), 1.41-1.54 (m, 4H), 2.01-2.09 (m, 8H), 2.59 (t, J=7.2 Hz, 4H), 2.77 (t, J=5.6 Hz, 4H), 5.28-5.43 (m, 8H)

### Reference Example 2

### Di((Z)-octadec-9-enyl)amine (Compound IIb-2)

Compound IIb-2 (0.562 g, 36.2%) was obtained in the same manner as in Reference Example 1 by using ammonia (manufactured by Tokyo Chemical Industry Co., Ltd., about 2 mol/L methanol solution, 12.0 mL, 24.0 mmol) and (Z)-octadec-9-enyl methanesulfonate (manufactured by Nu-Chek Prep, Inc., 1.87 g, 5.40 mmol).

ESI-MS m/z: 519 (M+H)⁺; ¹H-NMR (CDCl₃) δ: 0.88 (t, J=6.7 Hz, 6H), 1.29 (br s, 45H), 1.41-1.52 (m, 4H), 1.97-2.05 (m, 8H), 2.58 (t, J=7.2 Hz, 4H), 5.23-5.40 (m, 4H)

### Reference Example 3

### Di((Z)-hexadec-9-enyl)amine (Compound IIb-3)

Compound IIb-3 (0.243 g, 36.0 k) was obtained in the same manner as in Reference Example 1 by using ammonia (manufactured by SIGMA-ALDRICH Co., Ltd., about 7 mol/L methanol solution, 1.66 mL, 11.6 mmol) and (Z)-hexadec-9-enyl methanesulfonate (manufactured by Nu-Chek Prep, Inc., 0.488 g, 1.46 mmol). ¹H-NMR (CDCl₃) δ: 0.89 (t, J=6.9 Hz, 6H), 1.24-1.37 (m, 37H), 1.43-1.52 (m, 4H), 1.98-2.05 (m, 8H), 2.58 (t, J=7.2 Hz, 4H), 5.31-5.38 (m, 4H)

### Reference Example 4

### Di((11Z,14Z)-icosa-11,14-dienyl)amine (Compound IIb-4)

Compound IIb-4 (0.292 g, 36.6%) was obtained in the same manner as in Reference Example 1 by using ammonia (manufactured by SIGMA-ALDRICH Co., Ltd., about 7 mol/L methanol solution, 1.60 mL, 11.2 mmol) and (11Z,14Z)-icosa-11,14-dienyl methanesulfonate (manufactured by Nu-Chek Prep, Inc., 0.521 g, 1.40 mmol).

¹H-NMR (CDCl₃) δ: 0.89 (t, J=6.8 Hz, 6H), 1.24-1.39 (m, 41H), 1.43-1.51 (m, 4H), 2.02-2.08 (m, 8H), 2.58 (t, J=7.3 Hz, 4H), 2.77 (t, J=6.7 Hz, 4H), 5.30-5.41 (m, 8H)

### Example 1

### 3-(Dimethylamino)propyl di((9Z,12Z)-octadeca-9,12-dienyl)carbamate (Compound I-1)

Compound IIb-1 (1.35 g, 2.63 mmol) obtained in Reference Example 1 was dissolved in chloroform (18 mL), and 3-(dimethylamino)propyl 4-nitrophenyl carbonate hydrochloride (1.20 g, 3.94 mmol) synthesized according to the method described in “Journal of American Chemical Society (J. Am. Chem. Soc.)”, 1981, Vol. 13.03, pp. 4194-4199 and triethylamine (1.47 mL, 10.5 mmol) were added thereto, followed by heating and stirring at 110° C. for 60 minutes using a microwave reactor. To the reaction mixture, 3-(dimethylamino)propyl 4-nitrophenyl carbonate hydrochloride (200 mg, 0.658 mmol) was added, followed by heating and stirring at 110° C. for 20 minutes using a microwave reactor. The reaction mixture was diluted with chloroform, washed three times with a 1 mol/L sodium hydroxide aqueous solution and then washed with saturated brine, and thereafter dried over anhydrous magnesium sulfate. Subsequently, the resultant was filtered and concentrated under reduced pressure. The obtained residue was dissolved in a small amount of hexane/ethyl acetate (1/4), and the solution was adsorbed on an amino-modified silica gel pad. Then, the target material was eluted with hexane/ethyl acetate (1/4), and concentrated under reduced pressure. The resulting residue was purified by silica gel column chromatography (chloroform/methanol=100/0 to 95/5), whereby Compound I-1 (1.39 g, 82.2) was obtained.

ESI-MS m/z: 644 (M+H)⁺; ¹H-NMR (CDCl₃) δ: 0.89 (t, J=6.7 Hz, 6H), 1.29 (br s, 32H), 1.45-1.56 (m, 4H), 1.74-1.85 (m, 2H), 2.00-2.09 (m, 8H), 2.23 (s, 6H), 2.35 (t, J=7.4 Hz, 2H), 2.77 (t, J=5.8 Hz, 4H), 3.13-3.23 (m, 4H), 4.10 (t, J=6.4 Hz, 2H), 5.28-5.43 (m, 8H)

### Example 2

### 3-(Dimethylamino)propyl di((Z)-octadec-9-enyl)carbamate (Compound I-2)

Compound I-2 (0.267 g, 88.7%) was obtained in the same manner as in Example 1 by using Compound IIb-2 (0.156 g, 0.301 mmol) obtained in Reference Example 2 in place of Compound IIb-1.

ESI-MS m/z: 648 (M+H)⁺; ¹H-NMR (CDCl₃) δ: 0.88 (t, J=6.6 Hz, 6H), 1.28 (br s, 44H), 1.45-1.55 (m, 4H), 1.75-1.85 (m, 2H), 1.97-2.04 (m, 8H), 2.23 (s, 6H), 2.34 (t, J=7.6 Hz, 2H), 3.13-3.24 (m, 4H), 4.1.0 (t, J=6.4 Hz, 2H), 5.28-5.40 (m, 4H)

### Example 3

### 3-(Dimethylamino)propyl di((Z)-hexadec-9-enyl)carbamate (Compound I-3)

Compound I-3 (0.116 g, 55.2%) was obtained in the same manner as in Example 1 by using Compound IIb-3 (0.164 g, 0.355 mmol) obtained in Reference Example 3 in place of Compound IIb-1.

ESI-MS m/z: 592 (M+H)⁺; ¹H-NMR (CDCl₃) δ: 0.88 (t, J=6.9 Hz, 6H), 1.21-1.38 (m, 38H), 1.47-1.54 (m, 4H), 1.75-1.83 (m, 2H), 2.00-2.04 (m, 8H), 2.22 (s, 6H), 2.34 (t, J=7.4 Hz, 2H), 3.11-3.24 (m, 4H), 4.10 (t, J=6.4 Hz, 2H), 5.30-5.38 (m, 4H)

### Example 4

### 3-(Dimethylamino)propyl di((11Z,14Z)-icosa-11,14-dienyl)carbamate (Compound I-4)

Compound I-4 (0.290 g, 82.2%) was obtained in the same manner as in Example 1 by using Compound IIb-4 (0.288 g, 0.505 mmol) obtained in Reference Example 4 in place of Compound IIb-1.

ESI-MS m/z: 700 (M+H)⁺; ¹H-NMR (CDCl₃) δ: 0.89 (t, J=6.8 Hz, 6H), 1.21-1.40 (m, 40H), 1.46-1.54 (m, 4H), 1.76-1.83 (m, 2H), 2.02-2.08 (m, 8H), 2.23 (s, 6H), 2.35 (t, J=7.6 Hz, 2H), 2.77 (t, J=6.7 Hz, 4H), 3.10-3.24 (m, 4H), 4.10 (t, J=6.4 Hz, 2H), 5.30-5.41 (m, 8H)

### Example 5

### 2-(Dimethylamino)ethyl di((9Z,12Z)-octadeca-9,2-dienyl)carbamate (Compound I-5)

Compound I-5 (0.184 g, 70.0%) was obtained in the same manner as in Example 1 by using Compound IIb-1 (0.215 g, 0.418 mmol) obtained in Reference Example 1 and 2-(dimethylamino)ethyl 4-nitrophenyl carbonate hydrochloride (0.162 g, 0.557 mmol) in place of 3-(dimethylamino)propyl 4-nitrophenyl carbonate hydrochloride.

ESI-MS m/z: 630 (M+H)⁺; ¹H-NMR (CDCl₃) δ: 0.89 (t, J=6.8 Hz, 6H), 1.12-1.39 (m, 32H), 1.45-1.54 (m, 4H), 2.00-2.07 (m, 8H), 2.28 (s, 6H), 2.57 (t, J=7.2 Hz, 2H), 2.77 (t, J=6.7 Hz, 4H), 3.11-3.24 (m, 4H), 4.17 (t, J=6.7 Hz, 2H), 5.28-5.41 (m, 8H)

### Reference Example 5

### 5-Amino-N,N-di((9Z,12Z)-octadeca-9,12-dienyl)pentanamide (Compound VII-1)

Compound IIb-(1.50 mg, 0.292 mmol) obtained in Reference Example 1 was dissolved in chloroform (4 mL), and 5-(tert-butoxycarbonylamino)pentanoic acid (manufactured by Tokyo Chemical Industry Co., Ltd., 95 mg, 0.438 mmol), diisopropylethylamine (0.255 mL, 1.46 mmol), and HATU (O-(7-azabenzotriazol-1-yl)-N,N,N′,N′-tetramethyluronium hexafluorophosphate) (manufactured by Aldrich Co., Ltd., 222 mg, 0.584 mmol) were added thereto, followed by stirring at room temperature for 4 hours. To the reaction mixture, a saturated sodium hydrogen carbonate aqueous solution was added, and the aqueous layer was extracted with ethyl acetate. The organic layer was washed successively with water and saturated brine, and dried over anhydrous magnesium sulfate. Thereafter, the resultant was filtered and concentrated under reduced pressure. The obtained residue was purified by silica gel column chromatography (chloroform/methanol=100/0 to 97/3), whereby tert-butyl 5-(di((9Z,12Z)-octadeca-9,12-dienyl)amino)-5-oxopentylcarbamate was obtained.

The obtained tert-butyl 5-(di((9Z,12Z)-octadeca-9,12-dienyl)amino)-5-oxopentylcarbamate was dissolved in dichloromethane (4 mL), and trifluoroacetic acid (0.450 mL, 5.84 mmol) was added thereto, followed by stirring at room temperature for 4 hours. To the reaction mixture, a saturated sodium hydrogen carbonate aqueous solution was added, and the aqueous layer was extracted with chloroform. The organic layer was washed with saturated brine, and dried over anhydrous magnesium sulfate. Thereafter, the resultant was filtered and concentrated under reduced pressure. The obtained residue was purified by silica gel column chromatography (chloroform/methanol=100/0 to 90/10), whereby Compound VII-1 (124 mg, 96.1% in 2 steps) was obtained. ESI-MS m/z: 614 (M+H)⁺; ¹H-NMR (CDCl₃) δ: 0.89 (t, J=6.8 Hz, 6H), 1.28-1.38 (m, 32H), 1.43-1.57 (m, 6H), 1.63-1.73 (m, 2H), 2.05 (q, J=7.0 Hz, 8H), 2.30 (t, J=7.2 Hz, 2H), 2.71 (t, J=7.2 Hz, 2H), 2.77 (t, J=6.2 Hz, 4H), 3.1.9 (t, J=7.7 Hz, 2H), 3.28 (t, J=7.7 Hz, 2H), 5.28-5.43 (m, 8H)

### Reference Example 6

### 5-(Dimethylamino)-N,N-di((9Z,12Z)-octadeca-9,12-dienyl)pentanamide (Compound VII-2)

Compound VII-1 (90.0 mg, 0.147 mmol) obtained in Reference Example 5 was dissolved in 1,2-dichloroethane (2 mL) and methanol (2 mL), and formaldehyde (0.219 mL, 2.94 mmol) and sodium triacetoxyborohydride (manufactured by Acros Organics, 311 mg, 1.47 mmol) were added thereto, followed by stirring at room temperature for 5 hours. To the reaction mixture, a saturated sodium hydrogen carbonate aqueous solution was added, and the aqueous layer was extracted with ethyl acetate. The organic layer was washed with a saturated sodium chloride aqueous solution, and dried over anhydrous magnesium sulfate. Thereafter, the resultant was filtered and concentrated under reduced pressure. The obtained residue was purified by silica gel column chromatography (chloroform/methanol 100/0 to 75/25), whereby Compound VII-2 (88.2 mg, 93.9%) was obtained.

ESI-MS m/z: 642 (M+H)⁺; ¹H-NMR (CDCl₃) δ: 0.89 (t, J=7.0 Hz, 6H), 1.26-1.38 (m, 32H), 1.46-1.71 (m, 8H), 2.05 (q, J=7.0 Hz, 8H), 2.22 (s, 6H), 2.29 (q, J=7.0 Hz, 4H), 2.77 (t, J=6.2 Hz, 4H), 3.19 (t, J=7.9 Hz, 2H), 3.28 (t, J=7.7 Hz, 2H), 5.28-5.42 (m, 8H)

### Reference Example 7

### 3-Aminopropyl di((9Z,12Z)-octadeca-9,12-dienyl)carbamate (Compound VII-3)

Compound IIb-1 (146 mg, 0.284 mmol) obtained in Reference Example 1 was dissolved in N,N-dimethylformamide (5 mL), and tert-butyl 3-((4-nitrophenoxy)carbonyloxy)propylcarbamate (145 mg, 0.426 mmol) synthesized according to the method described in “Journal of American Chemical Society (J. Am. Chem. Soc.)”, 1981, Vol. 103, pp. 4194-4199 and triethylamine (0.158 mL, 1.14 mmol) were added thereto, followed by stirring overnight at room temperature. To the reaction mixture, water was added, and the aqueous layer was extracted with ethyl acetate. The organic layer was washed successively with water and saturated brine, and dried over anhydrous magnesium sulfate. Thereafter, the resultant was filtered and concentrated under reduced pressure. The obtained residue was purified by silica gel column chromatography (chloroform/methanol=100/0 to 98/2), whereby 3-((N-butoxycarbonyl)amino)propyl di((9Z,12Z)-octadeca-9,12-dienyl)carbamate was obtained.

The obtained 3-((N-butoxycarbonyl)amino)propyl di((9Z,12Z)-octadeca-9,12-dienyl)carbamate was dissolved in dichloromethane (4 mL), and trifluoroacetic acid (0.242 mL, 3.13 mmol) was added thereto, followed by stirring at room temperature for 8 hours. To the reaction mixture, a saturated sodium hydrogen carbonate aqueous solution was added, and the aqueous layer was extracted with chloroform. The organic layer was washed with saturated brine, and dried over anhydrous magnesium sulfate. Thereafter, the resultant was filtered and concentrated under reduced pressure. The obtained residue was purified by silica gel column chromatography (NH silica gel, chloroform/methanol=100/0 to 90/10), whereby Compound VII-3 (75.6 mg, 43.31 in 2 steps) was obtained.

ESI-MS m/z: 616 (M+H)⁺; ¹H-NMR (CDCl₃) δ: 0.89 (t, J=6.8 Hz, 6H), 1.26-1.38 (m, 32H), 1.46-1.54 (m, 4H), 1.73-1.82 (m, 2H), 2.05 (q, J=6.6 Hz, 8H), 2.76-2.80 (m, 6H), 3.18 (br s, 4H), 4.15 (t, J=6.2 Hz, 2H), 5.29-5.43 (m, 8H)

### Reference Example 8

### 2-(1-Methylpyrrolidin-2-yl)ethyl 4-nitrophenyl carbonate hydrochloride (Compound VI-1)

To a solution of 4-nitrophenyl chloroformate (manufactured by Tokyo Chemical Industry Co., Ltd., 1.761 g, 8.56 mmol) in diethyl ether (20 mL), a solution of 2-(1-methylpyrrolidin-2-yl)ethanol (manufactured by Tokyo Chemical Industry Co., Ltd., 1.0 mL, 7.13 mmol) in diethyl ether (20 mL) was added, followed by stirring overnight at room temperature. The reaction mixture was concentrated under reduced pressure, and the obtained residue was crystallized from ethanol/diethyl ether (1/1), followed by filtration, whereby Compound VI-1 (1.27 g, 54%) was obtained.

¹H-NMR (DMSO-d₆) δ: 1.59-1.77 (m, 2H), 1.82-2.09 (m, 3H), 2.15-2.26 (m, 1H), 2.76 (s, 3H), 2.93-3.05 (m, 2H), 3.61-3.20 (m, 3H), 4.80 (br s, 1H), 6.95 (d, J=9.2 Hz, 2H), 8.11 (d, J=9.2 Hz, 2H)

### Reference Example 9

### 4-Nitrophenyl 3-(piperidin-1-yl)propyl carbonate hydrochloride (Compound VI-2)

To a solution of 4-nitrophenyl carbonochloridate (1.58 g, 7.67 mmol) in diethyl ether (32 mL), 3-(piperidin-1-yl)propan-1-ol (manufactured by Aldrich Co., Ltd., 1.00 mL, 6.39 mmol) was added, followed by stirring overnight at room temperature. The reaction mixture was concentrated under reduced pressure, and the obtained residue was crystallized from ethanol, followed by filtration, whereby Compound VI-2 (1.86 g, 84%) was obtained.

ESI-MS m/z: 309 (M+H); ¹H-NMR (DMSO-d₆) δ: 1.28-1.49 (m, 1H), 1.62-1.89 (m, 5H), 2.10-2.26 (m, 2H), 2.76-2.96 (m, 2H), 3.04-3.19 (m, 2H), 3.36-3.49 (m, 2H), 4.33 (t, J=6.1 Hz, 2H), 7.58 (d, J=9.2 Hz, 2H), 8.33 (d, J=9.2 Hz, 2H), 10.37 (br s, 1H)

### Reference Example 10

### 4-Nitrophenyl 3-(pyrrolidin-1-yl)propyl carbonate hydrochloride (Compound VI-3)

To a solution of 4-nitrophenyl chloroformate (596 mg, 2.84 mmol) in diethyl ether (10 mL), a solution of 3-(pyrrolidin-1-yl)propan-1-ol (manufactured by ABCR, Inc., 386 mg, 2.84 mmol) in diethyl ether (10 mL) was added, followed by stirring at room temperature for 2 hours. The reaction mixture was concentrated under reduced pressure, and the obtained residue was crystallized from ethanol, followed by filtration, whereby Compound VI-3 (498 mg, 53%) was obtained. ¹H-NMR (DMSO-d₆) δ: 1.76-1.84 (m, 2H), 1.85-2.00 (m, 4H), 3.11-3.16 (m, 2H), 3.30-3.44 (m, 4H), 3.47 (t, J=6.0 Hz, 2H), 4.77 (br s, 1H), 6.95 (d, J=9.2 Hz, 2H), 8.11 (d, J=9.2 Hz, 2H)

### Example 6

### 2-(1-Methylpyrrolidin-2-yl)ethyl di((9Z,12Z)-octadeca-9,12-dienyl)carbamate (Compound I-6)

Compound IIb-1 (0.161 g, 0.314 mmol) obtained in Reference Example 1 was dissolved in acetonitrile (3.0 mL), and Compound VI-1 (0.156 g, 0.470 mmol) obtained in Reference Example 8 and triethylamine (0.219 mL, 1.57 mmol) were added thereto, followed by stirring at 80° C. for 2 hours. The reaction mixture was diluted with ethyl acetate, washed with water, and dried over anhydrous magnesium sulfate. Thereafter, the resultant was filtered and concentrated under reduced pressure. The obtained residue was purified by silica gel column chromatography (n-hexane/ethyl acetate=80/20), whereby Compound I-6 (0.172 g, 82%) was obtained.

ESI-MS m/z: 670 (M+H)⁺; ¹H-NMR (CDCl₃) δ: 0.89 (t, J=6.9 Hz, 6H), 1.20-1.40 (m, 32H), 1.45-1.57 (m, 6H), 1.62-1.83 (m, 2H), 1.94-2.18 (m, 12H), 2.31 (s, 3H), 2.77 (dd, J=6.8, 6.5 Hz, 2H), 3.03-3.26 (m, 4H), 4.06-4.17 (m, 2H), 5.29-5.42 (m, 8H)

### Example 7

### 3-(Piperidin-1-yl)propyl di((9Z,12Z)-octadeca-9,12-dienyl)carbamate (Compound I-7)

Compound I-7 (0.387 g, 81%) was obtained in the same manner as in Example 1 by using Compound VI-2 obtained in Reference Example 9 in place of 3-(dimethylamino)propyl 4-nitrophenyl carbonate hydrochloride.

ESI-MS m/z: 684 (M+H)⁺; ¹H-NMR (CDCl₃) δ: 0.89 (t, J=6.9 Hz, 6H), 1.21-1.62 (m, 42H), 1.79-1.86 (m, 2H), 2.02-2.08 (m, 8H), 2.32-2.42 (m, 6H), 2.77 (dd, J=6.8, 6.6 Hz, 2H), 3.10-3.43 (m, 4H), 4.09 (t, J=6.4 Hz, 1H), 5.29-5.42 (m, 8H)

### Example 8

### 3-(Pyrrolidin-1-yl)propyl di((9Z,12Z)-octadeca-9,2-dienyl)carbamate (Compound I-8)

Compound I-8 (0.225 g, 99%) was obtained in the same manner as in Example 6 by using Compound VI-3 (0.168 g, 0.508 mmol) obtained in Reference Example 10 in place of Compound VI-1.

ESI-MS m/z: 670 (M+H)⁺; ¹H-NMR (CDCl₃) δ: 0.89 (t, J=6.9 Hz, 6H), 1.21-1.40 (m, 32H), 1.46-1.55 (m, 4H), 1.76-1.80 (m, 2H), 1.82-1.89 (m, 2H), 2.01-2.08 (m, 8H), 2.47-2.55 (m, 6H), 2.77 (dd, J=6.7, 6.7 Hz, 2H), 3.11-3.24 (m, 4H), 4.11 (t, J=6.4 Hz, 2H), 5.29-5.42 (m, 8H)

### Example 9

Compositions were prepared as follows by using the compounds (Compounds I-1 to I-5) obtained in Examples 1 to 5. The used nucleic acid is an anti-APO-B siRNA, which suppresses the expression of an apolipoprotein-E (hereinafter, represented by “apo-b”) gene, and is composed of the base sequence of a sense strand [5′-rGmUrCrAmUrCrArCrArCmUrGrArAmUrArCrCrArAmU-3′ (the sugars attached to the bases marked with r are riboses, and marked with m are riboses having —O-methyl substituted for the hydroxyl group at the 2′ position)] and the base sequence of an antisense strand [5′-rArUzUrGrGrUrArUrUrCrArGrUrGrUrGrArUrGrArCrArC-3′ (all the sugars attached to the bases are riboses, and the 5′ end is phosphorylated)], and was obtained from Gene Design, Inc. (hereinafter referred to as “apo-b siRNA”).

Each sample was weighed so that the ratio of each of Compounds I-1 to I-5/1,2-dimyristoyl-sn-glycero-3-phosphoethanolamine-N-(methoxy(polyethylene glycol)-2000) sodium salt (PEG-DMPE Na, N-(carbonylmethoxypolyethylene glycol 2000)-1,2-dimyristoyl-sn-glycero-3-phosphoethanolamine sodium salt, manufactured by NOF Corporation)/disteazoylphosphatidyl choline (DSPC, 1,2-distearoyl-sn-glycero-3-phosphocholine, manufactured by NOF Corporation)/choiestercl (manufactured by NOF Corporation)=8.947/1.078/5.707/13.698 mmol/L, and dissolved in 90 vol % ethanol, whereby a solution containing the constituent components of a lipid membrane was prepared. Separately, apo-b siRNA in distilled water (24 mg/mL) was diluted with a Tris-EDTA buffer solution (200 mM Tris-HCl, 20 mM EDTA, manufactured by Invitrogen Co., Ltd.) and a 20 mM citric acid buffer solution (pH 5.0), whereby a 1.5 mg/mL apo-b siRNA aqueous solution (2 mM Tris-EDTA buffer solution, 20 mM citric acid buffer solution, pH 5.0) was prepared.

The obtained lipid solution was heated to 37° C., and a 500 μL portion was transferred to a container for preparing a preparation. The obtained apo-b siRNA aqueous solution (500 μL) was then added thereto while stirring. Then, a 20 mM citric acid buffer solution (containing 300 mM NaCl, pH 6.0, 1000 μL) was added to the obtained lipid nucleic acid mixed suspension (1000 μL) while stirring, and further, DPBS (manufactured by Invitrogen Co., Ltd., 3310 μL) was added dropwise thereto, whereby a crude preparation was obtained. The obtained crude preparation was concentrated by using Amicon Ultra (manufactured by Millipore Co., Ltd.), and further, the solvent was replaced with DPBS, and the resulting mixture was filtered through a 0.2-μm filter (manufactured by Toyo Roshi Kaisha, Ltd.) in a laminar flow cabinet. Further, the siRNA concentration in the obtained preparation was measured, and the preparation was diluted with DPBS so that the siRNA concentration was 0.3 mg/mL, whereby preparations (compositions containing any of Compounds I-1 to I-5, and the nucleic acid) were obtained.

The average particle diameter of the lipid nanoparticles in each preparation was measured using a particle diameter measurement device (Zetasizer Nano ZS, manufactured by Malvern). The results are shown in Table 3.

TABLE 3

| | Compound No. | | | | |
|---|---|---|---|---|---|
| | I-1 | I-2 | I-3 | I-4 | I-5 |
| Particle diameter of | 151.8 | 135.8 | 150.0 | 159.0 | 137.0 |
| Preparation obtained (nm) | | | | | |

### Comparative Example 1

Preparations were obtained in the same manner as in Example 9 except that Compounds I-1 to I-5 were changed to DLin-KC2-DMA synthesized by a modified method of the method described in WO2010/042877 and the compounds obtained in Reference Examples 5 to 7 (Compounds VII-1 to VII-3).

The structural formulae of the compounds (DLin-KC2-DMA and Compounds VII-1 to VII-3) used in the comparative example are shown in Table 4.

The average particle diameter of the lipid nanoparticles in each preparation was measured using a particle diameter measurement device. The results are shown in Table 5.

TABLE 4

| Compound No. | Structural formula |
|---|---|
| DLin-KC2-DMA | 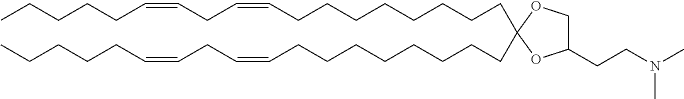 [SMILES: CCCCC/C=C\C/C=C\CCCCCCCCC1(CCCCCCCC/C=C\C/C=C\CCCCC)OCC(CCN(C)C)O1] |
| VII-1 | 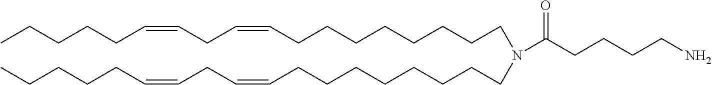 [SMILES: CCCCC/C=C\C/C=C\CCCCCCCCN(CCCCCCCC/C=C\C/C=C\CCCCC)C(=O)CCCCN] |
| VII-2 | 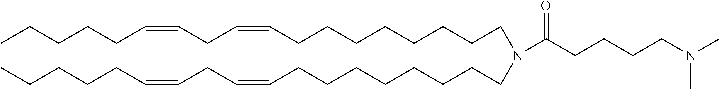 [SMILES: CCCCC/C=C\C/C=C\CCCCCCCCN(CCCCCCCC/C=C\C/C=C\CCCCC)C(=O)CCCCN(C)C] |
| VII-3 | 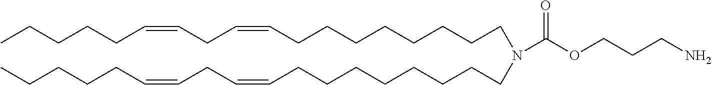 [SMILES: CCCCC/C=C\C/C=C\CCCCCCCCN(CCCCCCCC/C=C\C/C=C\CCCCC)C(=O)OCCCN] |

TABLE 5

| | Compound No. | | | |
|---|---|---|---|---|
| | DLin-KC2-DMA | VII-1 | VII-2 | VII-3 |
| Particle diameter of | 159.8 | 176.6 | 160.1 | 177.7 |
| Preparation obtained (nm) | | | | |

### Test Example 1

Each of the preparations obtained in Example 9 (the compositions containing any of Compounds I-1, and I-3 to I-5, and the nucleic acid) and the preparations obtained in Comparative Example 1 (the compositions containing any of DLin-KC2-DMA and Compounds VII-1 to VII-3, and the nucleic acid) was introduced into cells of a human liver cancer-derived cell line HepG2 (HB-8065) by the following method.

Each preparation diluted with Opti-MEM (GIBCO Co., Ltd., 31985) so that the final concentration of the nucleic acid was 1 to 30 nM was dispensed in a 96-well culture plate at 20 μL/well. Then, HepG2 cells suspended in MEM containing 1.25% fetal bovine serum (FBS, SAFC Biosciences, Inc., 12203C) were inoculated at 6250 cells/80 μL/well, and cultured under the conditions of 37° C. and 5% CO₂, thereby introducing the preparation into the HepG2 cells. Further, untreated cells were inoculated as a negative control group.

The cells after the introduction of the preparation were cultured in a 5% CO₂ incubator at 37° C. for 24 hours, and then washed with ice-cooled phosphate buffered saline (PBS, GIBCO Co., Ltd., 14190). Thereafter, by using a Cells-to-Ct Kit (Applied Biosystems (ABI), Inc., AM1728), the total RNA was collected, and cDNA was synthesized by a reverse transcription reaction using the obtained total RNA as a template according to the method described in the protocol attached to the kit.

By using the obtained cDNA as a template and also using a universal probe library (Roche Applied Science, Inc., 04683633001) as the probe and ABI7900HT Fast (manufactured by ABI, Inc.), a PCR reaction was performed according to the method described in the protocol attached thereto, so that the apo-b gene and D-glyceraldehyde-3-phosphate dehydrogenase (hereinafter, represented by “gapdh”) gene, which is a constitutively expressed gene, were subjected to the PCR reaction. Then, the amount of the amplified mRNA was measured for each gene, and aquasi-quantitative value of the apo-b mRNA was calculated using the amount of the amplified gapdh mRNA as the internal control. Further, the amount of the apo-b mRNA and the amount of the amplified gapdh mRNA in the negative control group were measured in the same manner, and a quasi-quantitative value of the apo-b mRNA was calculated using the amount of the amplified gapdh mRNA as the internal, control.

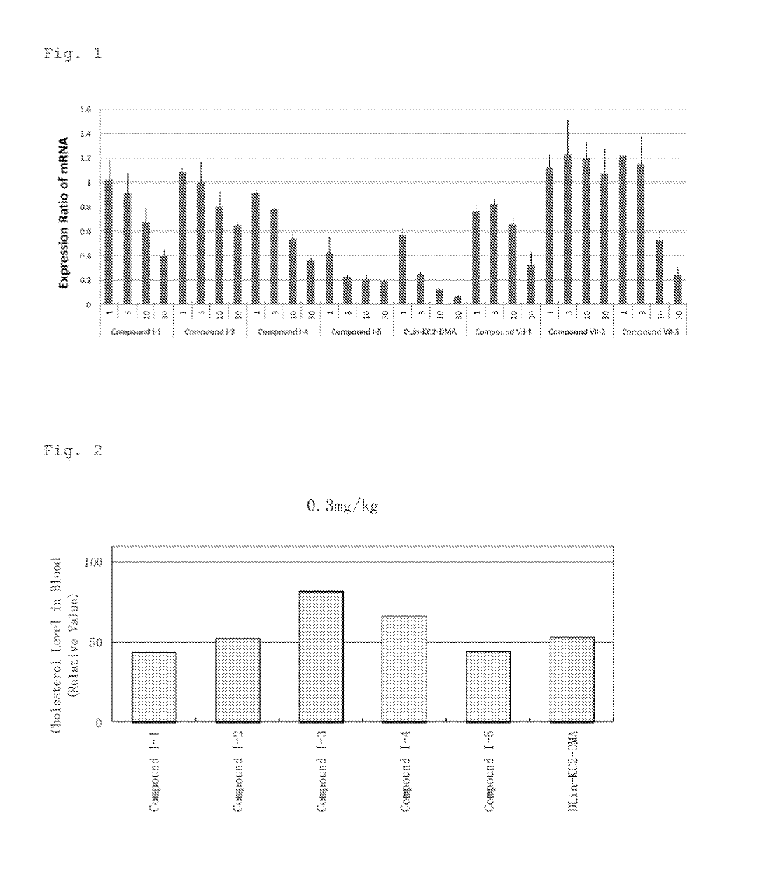

The expression ratio of the apo-b mRNA was determined from the calculated quasi-quantitative value of the apo-b mRNA with the quasi-quantitative value of the apo-b mRNA of the negative control taken as 1, and the results of the expression ratio are shown in FIG. 1.

As apparent from FIG. 1, the preparations obtained in Example 9 (the compositions containing any of Compounds I-1, and I-3 to I-5, and the nucleic acid), and among the preparations obtained in Comparative Example 1, the compositions containing DLin-KC2-DMA, Compound VII-1, or Compound VII-3, and the nucleic acid suppressed the expression of the apo-b gene mRNA after the introduction thereof into the cells of the human liver cancer-derived cell line HepG2. On the other hand, among the preparations obtained in Comparative Example 1, the composition containing Compound VII-2 and the nucleic acid did not suppress the expression of the apo-b gene mRNA after the introduction thereof into the cells of the human liver cancer-derived cell line HepG2.

### Test Example 2

Each of the preparations obtained in Example 9 (the compositions containing any of Compounds I-1 to I-5, and the nucleic acid) and the preparations obtained in Comparative Example 1 (the compositions containing any of DLin-KC2-DMA and Compounds VII-1 to VII-3, and the nucleic acid) was tested for evaluating the in vivo drug efficacy according to the following method. Incidentally, each preparation was used after it was diluted in accordance with the test.

After mice were housed and acclimated, each preparation was intravenously administered to mice at a dose of 3 or 0.3 mg/kg in terms of siRNA concentration. At 48 hours after the administration, blood was collected, and the collected blood was centrifuged at 3000 rpm for 20 minutes at 4° C. using a refrigerated microcentrifuge (05PR-22, manufactured by Hitachi, Ltd.). The cholesterol level in the thus obtained serum was determined as follows. A Cholesterol Assay Kit (Cat. No. 10007640, manufactured by Cayman Chemical Company) was used, and according to the method described in the protocol attached to the kit, the intensity of fluorescence was measured in a standard solution and in the serum sample using ARVO (530 nm/595 nm) or EnVision (531 nm/595 nm). On the basis of the obtained intensity of fluorescence, a calibration curve was prepared, and the cholesterol level in the serum was calculated.

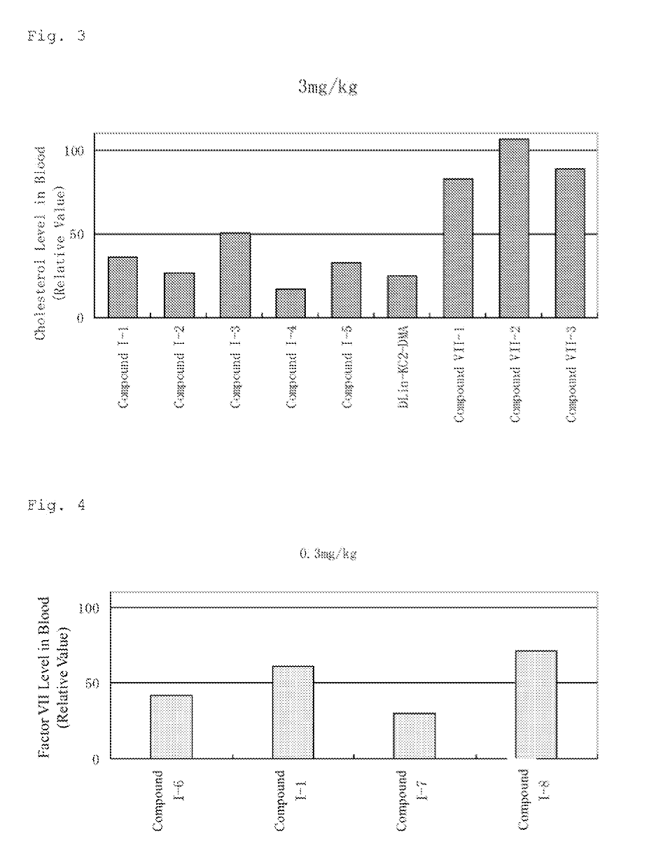

The results of the calculated cholesterol level in the serum are shown in FIGS. 2 and 3.

As apparent from FIGS. 2 and 3, the measurement results of the cholesterol level obtained by testing the preparations obtained in Example 9 (the compositions containing anti-APO-B siRNA which suppresses the expression of the apo-b gene, and any of Compounds I-1 to I-5) for evaluating the in vivo drug efficacy are lower as compared with the measurement results obtained by using the compositions containing any of Compounds VII-1 to VII-3, and the nucleic acid among the preparations obtained in Comparative Example 1, and it is shown that the expression of the apo-b gene is strongly suppressed.

Accordingly, it was revealed that the composition of the present invention can introduce a nucleic acid into a cell or the like, and the cationic lipid of the present invention is a cationic lipid which facilitates the in vivo delivery of a nucleic acid into a cell.

### Example 10

Compositions were prepared as follows by using the compound (Compound I-6) obtained in Example 6. The used nucleic acid is an anti-f7 siRNA, which suppresses the expression of a coagulation factor VII (hereinafter, represented by “f7”) gene, and is composed of the base sequence of a sense strand [5′-rGrGrAfUfCrAfUfCfUfCrArArGfUfCfUfUrAfCdTdT-3′ (the sugars attached to the bases marked with r are riboses, marked with d are deoxyriboses, and marked with f are riboses having fluorine substituted for the hydroxyl group at the 2′ position, and a bond between the deozyribose attached to the base at the position 20 from the 5′ end side to the 3′ end side and the deoxyribose attached to the base at the position 21 is a phosphorothioate bond)] and the base sequence of an antisense strand [5′-rGfUrArArGrAfCfUfUrGrArGrAfUrGrAfUfCfCdTdT-3′ (the sugars attached to the bases marked with r are riboses, marked with d are deoxyriboses, and marked with f are riboses having fluorine substituted for the hydroxyl group at the 2′ position, and a bond between the deoxyribose attached to the base at the position 20 from the 5′ end side to the 3′ end side and the deoxyribose attached to the base at the position 21 is a phosphorothioate bond)], and was obtained from Gene Design, Inc. (hereinafter referred to as “f7 siRNA”).

Each sample was weighed so that the ratio of Compound I-6/PEG-DMPE Na (manufactured by NOF Corporation)/DSPC (manufactured by NOF Corporation)/cholesterol (manufactured by NOF Corporation)=3.532/0.270/1.156/2.401 mmol/L, and dissolved in 100 volt ethanol, whereby a solution containing the constituent components of a lipid membrane was prepared.

Separately, f7 siRNA in distilled water (24 mg/mL) was diluted with a Tris-EDTA buffer solution (200 mM Tris-HCl, 20 mM EDTA, manufactured by Invitrogen Co., Ltd.) and a 20 mM citric acid buffer solution (pH 4.0), whereby a 0.375 mg/mL f7 siRNA aqueous solution (2 mM Tris-EDTA buffer solution, 20 mM citric acid buffer solution, pH 4.0) was prepared.

The obtained lipid solution was heated to 37° C., and an 800 μL portion was transferred to a container for preparing a preparation. The obtained f7 siRNA aqueous solution (800 μL) was then added thereto while stirring. Then, a 20 mM citric acid buffer solution (containing 300 mM NaCl, pH 6.0, 1600 μL) was added to the obtained lipid nucleic acid mixed suspension (1600 μL) while stirring, and further, DPBS (manufactured by Invitrogen Co., Ltd., 7086 L) was added dropwise thereto, whereby a crude preparation was obtained. The obtained crude preparation was concentrated by using Amicon Ultra (manufactured by Millipore Co., Ltd.), and further, the solvent was replaced with DPBS, and the resulting mixture was filtered through a 0.45-μm filter (manufactured by Toyo Roshi Kaisha, Ltd.) in a laminar flow cabinet. Further, the siRNA concentration in the obtained preparation was measured, and the preparation was diluted with DPBS so that the siRNA concentration was 0.03 mg/mL, whereby a preparation (a composition containing Compound I-6 and the nucleic acid) was obtained.

The average particle diameter of the lipid nanoparticles in the preparation was measured using a particle diameter measurement device. The results are shown in Table 6.

### Example 11

Preparations (compositions containing Compound I-1, I-7, or I-8, and the nucleic acid) were obtained in the same manner as in Example 10 by using a compound (Compound I-1, I-7, or I-8) obtained in Example 1, 7, or 8.

The average particle diameter of the lipid nanoparticles in each preparation was measured using a particle diameter measurement device. The results are shown in Table 6.

TABLE 6

Compound No.

| | I-6 | I-1 | I-7 | I-8 |
|---|---|---|---|---|
| Particle diameter of | 121.1 | 118.9 | 131.8 | 121.6 |
| Preparation obtained (nm) | | | | |

### Test Example 3

Each of the preparations obtained in Examples 10 and 11 (the compositions containing Compound I-6, I-1, I-7, or I-8, and the nucleic acid) was tested for evaluating the in vivo drug efficacy according to the following method. Incidentally, each preparation was used after it was diluted in accordance with the test.

After mice (Balb/c, obtained from CLEA Japan, Inc.) were housed and acclimated, each preparation was intravenously administered to mice at a dose of 0.3 or 0.1 mg/kg in terms of siRNA concentration. At 48 hours after the administration, blood was collected, and the collected blood was centrifuged at 8000 rpm for 8 minutes at 4° C. using a high speed refrigerated microcentrifuge (TOMY MX305, manufactured by Tomy Seiko Co., Ltd.). The Factor VII protein level in the thus obtained plasma sample was determined as follows. A BIOPHEN VII Kit (Cat. No. A221304, manufactured by ANIARA Company) was used, and according to the method described in the protocol attached to the kit, the absorbance was measured in a standard solution and in the plasma sample using ARVO (405 nm). On the basis of the obtained absorbance, a calibration curve was prepared, and the Factor VII protein level in the plasma was calculated. Incidentally, the number of mice in each group was set to 3.

The results of the calculated Factor VII protein level in the plasma are shown in FIG. 4.

As apparent from FIG. 4, the measurement results of the Factor VII protein level in the plasma obtained by testing each of the preparations obtained in Examples 10 and 11 (the compositions containing anti-Factor VII siRNA which suppresses the expression of the Factor VII gene, and Compound I-6, I-1, I-7, or I-8) for evaluating the in vivo drug efficacy show that the expression of the Factor VII gene is strongly suppressed.

Accordingly, it was revealed that the composition of the present invention can introduce a nucleic acid into a cell or the like, and the cationic lipid of the present invention is a cationic lipid which facilitates the in vivo delivery of a nucleic acid into a cell.

### Example 12

By using Compounds I-1 to I-8 obtained in Examples 1 to 8, compositions are prepared as follows.

As a nucleic acid, the same sequence as in Example 10 is used, and the nucleic acid is used after it is prepared at 24 mg/mL with distilled water.

Each of Compounds I-1 to I-8 and PEG-DMPE Na (manufactured by NOF Corporation) are suspended in an aqueous solution containing hydrochloric acid and ethanol so that the ratio of each of Compounds I-1 to I-8 to PEG-DMPE Na is 57.3/5.52 mmol/L, and then, the resulting mixture is repeatedly subjected to stirring using a vortex stirring mixer and heating, whereby a uniform suspension is obtained. This suspension is passed through a 0.2-μm polycarbonate membrane filter and thereafter passed through a 0.05-μm polycarbonate membrane filter under room temperature, whereby a dispersion liquid of lead particles is obtained. The average particle diameter of the obtained lead particles is measured using a particle diameter measuring apparatus to confirm that the average particle diameter is within the range from 30 nm to 100 nm. In the obtained dispersion liquid of lead particles, the f7 siRNA solution is mixed at a ratio of 3:1, and then, distilled water that is three times the amount is added thereto and mixed therewith, whereby a dispersion liquid of cationic lipid/double-stranded nucleic acid complex particles is prepared.

Separately, each sample is weighed so that the ratio of each of Compounds I-1 to I-8/PEG-DMPE Na (manufactured by NOF Corporation)/DSPC (manufactured by NOF Corporation)/cholesterol (manufactured by NOF Corporation)-8.947/1.078/5.707/13.698 mmol/L, and dissolved in 90 vol % ethanol, whereby a solution containing the constituent components of a lipid membrane is prepared.

The obtained solution containing the constituent components of a lipid membrane is heated and then mixed with the obtained dispersion liquid of cationic lipid/double-stranded nucleic acid complex particles at a ratio of 1:1, and the resulting mixture is further mixed with distilled water that is several times the amount, whereby a crude preparation is obtained.

The obtained crude preparation is concentrated by using Amicon Ultra (manufactured by Millipore Co., Ltd.), and further, the solvent is replaced with physiological saline (saline), and the resulting mixture is filtered through a 0.2-μm filter (manufactured by Toyo Roshi Kaisha, Ltd.) in a laminar flow cabinet. Further, the siRNA concentration in the obtained preparation is measured, and the preparation is diluted with saline so that the siRNA concentration is 0.03 mg/mL, whereby preparations (compositions containing any of Compounds I-1 to 1-8, and the nucleic acid) are obtained.

### INDUSTRIAL APPLICABILITY

By administering a composition containing the novel cationic lipid of the present invention and a nucleic acid to a mammal or the like, the nucleic acid can be easily introduced into, for example, a cell or the like.

### Sequence Listing Free Text

SEQ No. 1: siRNA sense strand
SEQ No. 2: siRNA antisense strand
SEQ No. 3: siRNA sense strand
SEQ No. 4: siRNA antisense strand
   Sequence Listing

1. A cationic lipid represented by formula (I): 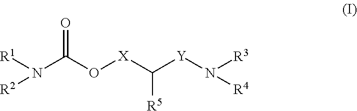
(wherein R¹ is linear or branched alkyl, alkenyl, or alkynyl, each having 8 to 24 carbon atoms,
R² is linear or branched alkyl, alkenyl, or alkynyl, each having 8 to 24 carbon atoms, or alkoxyethylene, alkoxypropylene, alkenyloxyethylene, alkenyloxypropylene, alkynyloxyethylene, or alkynyloxypropylene,
R³ and R⁴ may be the same or different, and are each alkyl having 1 to 3 carbon atoms or are combined together to form alkylene having 2 to 6 carbon atoms, or R³ and R⁵ are combined together to form alkylene having 2 to 6 carbon atoms,
R⁵ is a hydrogen atom, alkyl having 1 to 6 carbon atoms, alkenyl having 3 to 6 carbon atoms, amino, monoalkylamino, hydroxy, alkoxy, carbamoyl, monoalkylcarbamoyl, dialkylcarbamoyl, or alkyl having 1 to 6 carbon atoms or alkenyl having 3 to 6 carbon atoms, substituted with one to three of the same or different substituents selected from amino, monoalkylamino, hydroxy, alkoxy, carbamoyl, monoalkylcarbamoyl, and alkylcarbamoyl, or is combined together with R³ to form alkylene having 2 to 6 carbon atoms,
X is alkylene having 1 to 6 carbon atoms, and
Y is a single bond or alkylene having 1 to 6 carbon atoms, provided that the sum of the number of carbon atoms in X and Y is 6 or less, and when R⁵ is a hydrogen atom, Y is a single bond, and when R⁵ and R³ are combined together to form alkylene having 2 to 6 carbon atoms, Y is a single bond, or methylene or ethylene).

2. The cationic lipid according to claim 1, wherein R¹ and R² are tetradecyl, hexadecyl, (Z)-tetradec-9-enyl, (Z)-hexadec-9-enyl, (Z)-octadec-6-enyl, (Z)-octadec-9-enyl, (E)-octadec-9-enyl, (Z)-octadec-11-enyl, (9Z,12Z)-octadeca-9,12-dienyl, (9Z,12Z,15Z)-octadeca-9,12,15-trienyl, (Z)-icos-11-enyl, (11Z,14Z)-icosa-1,14-dienyl, 3,7,11-trimethyldodeca-2,6,10-trienyl, or (Z)-docos-13-enyl.

3. The cationic lipid according to claim 1, wherein R¹ and R² are hexadecyl, (Z)-hexadec-9-enyl, (Z)-octadec-6-enyl, (Z)-octadec-9-enyl, (9Z,12Z)-octadeca-9,12-dienyl, (Z)-icos-11-enyl, or (11Z,14Z)-icosa-11,14-dienyl.

4. The cationic lipid according to claim 1, wherein X is alkylene having 1, 2, or 3 carbon atoms, and Y is a single bond or methylene.

5. The cationic lipid according to claim 1, wherein R³ and R⁴ may be the same or different, and are each methyl or ethyl, or are combined together to form butylene or pentylene.

6. The cationic lipid according to claim 1, wherein R³ and R⁵ are combined together to form propylene, butylene, or pentylene, and R⁴ is methyl or ethyl.

7. A composition comprising the cationic lipid described in claim 1 and a nucleic acid.

8. The composition according to claim 7, wherein the cationic lipid forms a complex between the cationic lipid and the nucleic acid, or forms a complex between a combination of a neutral lipid and/or a polymer with the cationic lipid and the nucleic acid.

9. The composition according to claim 7, wherein the cationic lipid forms a complex between the cationic lipid and the nucleic acid, or forms a complex between a combination of a neutral lipid and/or a polymer with the cationic lipid and the nucleic acid, and the composition comprises the complex and a lipid membrane which encapsulates the complex.

10. The composition according to claim 7, wherein the nucleic acid is a nucleic acid which has an activity of suppressing the expression of a target gene by utilizing RNA interference (RNAi).

11. The composition according to claim 10, wherein the target gene is a gene which is expressed in the liver, lung, kidney, or spleen.

12. A method for introducing the nucleic acid described in claim 7 into a cell by using the composition described in claim 7.

13. The method according to claim 12, wherein the cell is a cell which is present in the liver, lung, kidney, or spleen of a mammal.

14. The method according to claim 12, wherein the method for introduction into a cell is a method for introduction into a cell by intravenous administration.

15. A method for treating a disease associated with the liver, lung, kidney, or spleen, comprising administering the composition described in claim 11 to a mammal.
